(19) United States
(12) Patent Application Publication
(10) Pub. No.: US 2016/0193296 A1
(43) Pub. Date: Jul. 7, 2016

(54) METHODS FOR TREATMENT OF MICROCEPHALY ASSOCIATED AUTISM DISORDERS

(71) Applicant: BROWN UNIVERSITY, Providence, RI (US)

(72) Inventor: Eric MORROW, Barrington, RI (US)

(21) Appl. No.: 14/654,232

(22) PCT Filed: Dec. 19, 2013
(86) PCT No.: PCT/US13/76609

§ 371 Date: Jun. 19, 2015

Related U.S. Application Data
(60) Provisional application No. 61/739,351, filed on Dec. 19, 2012.

(51) Int. Cl. A61K 38/18; A61K 38/12; A61K 31/366; A61K 31/353; C07K 16/40; A61K 31/351; C12N 7/00; A61K 31/365; A61K 31/4706; A61K 31/427; A61K 31/7052; A61K 38/06; A61K 35/545

(52) U.S. Cl. CPC A61K 38/185; A61K 38/06; A61K 38/12; A61K 31/366; A61K 31/353; C07K 16/40; A61K 35/545; C12N 7/00; A61K 31/365; A61K 31/4706; A61K 31/427; A61K 31/7052; A61K 31/351; C12N 2750/14143; C12N 2740/10043; C12N 2740/15043; C12N 2710/24043; C12N 2760/20143; C12N 2710/16743; C12N 2710/16643; C12N 2740/16043

(57) ABSTRACT

The invention features methods for the treatment of one or more symptoms of microcephaly associated autism disorders e.g., Christianson syndrome, by administering agents that increase the level or activity of BDNF or TrkB in the brain of the patient. These agents may include BDNF, a BDNF agonist, a BDNF mimetic, a TrkB agonist, a cell expressing recombinant BDNF, a BDNF encoding recombinant nucleic acid molecule encapsidated within a recombinant virus, and an agent that decreases the acidity of endosomes.

### CROSS-REFERENCE TO RELATED APPLICATIONS

This application claims the benefit of U.S. Provisional Application No. 61/739,351, filed Dec. 19, 2012, which is hereby incorporated by reference in its entirety.

### STATEMENT AS TO FEDERALLY FUNDED RESEARCH

This invention was made with government support under COBRE P20 RR018728-01, awarded by the National Institutes of Health. The government has certain rights in the invention.

### FIELD OF INVENTION

The invention is related to the field of treatment of neuro-developmental disorders such as Christianson syndrome and Angelman-like syndrome.

### BACKGROUND OF THE INVENTION

Intellectual and developmental disabilities (IDD) affect an estimated 4.6 million people in the US with annual costs in the range of $386 billion. There are few bio-therapeutic options to improve cognitive or functional gains for children with IDD, and this is an area of active research. Accumulating evidence indicates that a hallmark of a subset of IDD is altered axonal and dendritic growth and branching. Axonal branching phenotypes in postmortem studies in autism further support this hypothesis in human brain and in the more common idiopathic forms of IDD.

Postnatal microcephaly or macrocephaly, brain undergrowth or overgrowth respectively, observed in IDD and in several autistic disorders, also implicates axonal and dendritic growth and arborization as a potential mechanism in IDD. One such microcephaly associated autism disorder is Christianson syndrome. Therapeutics that target axonal and dendritic growth provide a plausible approach for potential interventions in microcephaly associated autistic disorders such as Christianson syndrome. Thus there is a need for the development of methods for treatment of microcephaly associated autistic disorders that may function by modulation of axonal and dendritic growth.

### SUMMARY OF THE INVENTION

The invention features methods of treating, ameliorating, reversing, or slowing the progression of at least one symptom of a microcephaly associated autism disorder in a subject. The methods of the present invention include administering an agent that increases the level or activity of brain derived neurotrophic factor (BDNF) and/or tyrosine related kinase B (TrkB) receptor in the brain of the subject, thereby treating, ameliorating, or slowing the progression of at least one symptom of a microcephaly associated autism disorder in the subject.

In certain embodiments, the microcephaly associated autism disorder is selected from the group inclusive of Christianson syndrome, Angelman-like syndrome, and autism disorders with microcephaly. In particular embodiments, the microcephaly associated autism disorder is Christianson syndrome. The symptoms of microcephaly associated autism disorder can be selected from the group inclusive of intellectual disability, such as an intelligence quotient (IQ) of 70 or less (e.g., an IQ of 70, 60, 50, 40, 30, 20, 10, or less), epilepsy, loss of speech, craniofacial dysmorphology, ataxia, difficulty with adaptive skills, difficulty walking, inability to walk, ophthalmoplegia, brain atrophy, autistic symptoms, and retinitis pigmentosa.

In some embodiments of the present invention, the subject has at least one mutation in the Nhe6 gene such that the gene encodes a mutant NHE6 protein, e.g., a mutant NHE6 protein that has an E255Q mutation or a mutant NHE6 protein has a D260N mutation.

The agent that increases the level or activity of BDNF or TrkB in the brain of the subject can be selected from the group inclusive of BDNF, a BDNF agonist, a BDNF mimetic, a TrkB agonist, a cell expressing recombinant BDNF, a BDNF encoding recombinant nucleic acid molecule encapsidated within a recombinant virus, and an agent that decreases the acidity of endosomes, i.e., an agent that decreases the acidity of endosomal compartments.

The agent for use in the methods of the present invention can be BDNF. In particular embodiments, the BDNF is purified naturally occurring BDNF or recombinant BDNF. In certain embodiments, the agent for use in the methods of the present invention is a BDNF agonist. In other embodiments the agent for use in the methods of the present invention is a BDNF mimetic, The BDNF mimetic that can be used in the methods of the invention can be a peptide mimetic including one or more mimetics selected from the group inclusive of a tricyclic dimeric peptide, a dipeptide, a tripeptide, and a cyclic pentapeptide. The tripeptide can be LM22A-1. The cyclic pentapeptide can be cyclic pentapeptide 2. Alternatively, the BDNF mimetic can be a chimeric neurotrophic factor. The BDNF mimetic can be a non-peptide mimetic or a small molecule mimetic. The small molecule mimetic can be selected from the group inclusive of LM22A-2, LM22A-3, and LM22A-4. The BDNF mimetic can also be deoxygedunin or 7, 8-dihydroxyflavone.

In certain embodiments, the BDNF agonist for use in the methods of the present invention is a TrkB activating antibody or a compound selected from the group inclusive of neurotrophin-4, neurotrophin-5, N-acetylserotonin, and 4′-dimethylamino-7, 8-dihydroxyflavone.

In some embodiments, the agent for use in the methods of the present invention can be a cell expressing BDNF, e.g., an induced pluripotent stem cell (iPSC) expressing BDNF, e.g., a recombinant BDNF. In other embodiments, the agent can be a recombinant virus in which a BDNF encoding recombinant nucleic acid molecule is encapsidated. The recombinant virus can be selected from the group inclusive of recombinant adeno-associated virus (AAV), recombinant retrovirus, recombinant lentivirus, recombinant poxvirus, recombinant rabies virus, recombinant pseudo-rabies virus, and recombinant herpes simplex virus, and human immunodeficiency virus (HIV).

In certain embodiments, the agent that increases the level or activity of brain derived neurotrophic factor (BDNF) and/or tyrosine related kinase B (TrkB) receptor in the brain of the subject is an agent that decreases the acidity of endosomes. In particular embodiments, the agent that decreases the acidity of endosomes is selected from the group inclusive of amantadine, amiodarone, ammonium chloride, azithromycin, bafilomycin A1, benzolactone enamides, bepridil, diphyllin, indolyls, macrolactones, monensin, nigericin, plecomacrolides, quinolines, or sulfonamides. In some embodiments, the agent that decreases the acidity of endosomes is a benzolactone enamide selected from the group inclusive of salicylihalamide, lobatamide, apicularen, oximidine, and cruentaren. In other embodiments, the agent that decreases the acidity of endosomes is an indole derivative of bafilomycin, e.g., INDOL0. In still other embodiments, the agent that decreases the acidity of endosomes is a macrolactone selected from the group inclusive of archazolid and azithromycin. In some embodiments, the agent that decreases the acidity of endosomes is a plecomacrolide selected from the group inclusive of bafilomycin A1 and concanamycin. In some embodiments, the agent that decreases the acidity of endosomes is a quinoline selected from the group inclusive of amodiaquine, chloroquine, and hydroxychloroquine. In particular embodiments, the quinoline is chloroquine. In other embodiments, the agent that decreases the acidity of endosomes is a sulfonamide selected from the group inclusive of 16D2 (5-bromo-2-{[(4-chloro-3-nitrophenyl)sulfonyl]amino}-N-(2,5-dichlorophenyl)benzamide) and 16D10 (5-chloro-2-{[(4-chloro-3-nitrophenyl)sulfonyl]amino}-N-(4-chlorophenyl)benzamide).

In any of the above methods, the symptom may be selected from the group inclusive of intellectual disability, an intelligence quotient (IQ) of 70 or less, epilepsy, inability to speak, craniofacial dysmorphology, ataxia, difficulty with adaptive skills, difficulty walking, inability to walk, ophthalmoplegia, brain atrophy, autistic symptoms, and retinitis pigmentosa. In such embodiments, the agent may treat, ameliorate, reverse, or slow progression toward the inability to speak or the inability to walk. The agent may treat, ameliorate, reverse, or slow progression of the intellectual disability and/or increase the IQ. The agent may decrease the subject's epilepsy or ataxia. The agent may treat, ameliorate, reverse, or slow progression of the subject's brain atrophy or difficulty walking. The agent may increase the adaptive skills of the subject.

Any of the above embodiments may further include, prior to administering the agent, testing the subject for microcephaly. In particular embodiments, the testing for microcephaly includes comparing the head circumference of the subject to that of a control subject or reference value.

Any of the above embodiments may further include, prior to administering the agent, testing the subject for the presence of a microcephaly associated autism disorder selected from the group including Christianson syndrome, Angelman-like syndrome, and autism with microcephaly. In particular embodiments, the testing may include testing the presence of one or more mutations in the Nhe6 gene in the subject relative to a control subject or reference sequence. In certain instances, the control subject is a subject without a microcephaly associated autism disorder. The testing may be or include a genomic sequencing assay, polymerase chain reaction assay, fluorescence in situ hybridization assay, or an immunoassay. The presence of mutations in the Nhe6 gene can be detected by a genomic sequencing assay, polymerase chain reaction assay, fluorescence in situ hybridization assay, or an immunoassay.

Any of the above embodiments may further include, prior to administering the agent, assaying in the subject one or more symptoms selected from the group inclusive of intellectual disability, delayed development, sleep disturbance, epilepsy, jerky movements (especially hand-flapping), social-interaction difficulties, communication challenges, repetitive behaviors, ataxia, craniofacial dysmorphology, difficulty with adaptive skills, difficulty standing or walking, inability to walk, ophthalmoplegia, brain atrophy, retinitis pigmentosa, severe limitation of speech and language, easily provoked laughter, a happy demeanor with frequent smiling or spontaneous laughter, impaired ocular movement, and autistic behaviors as measured by the autism diagnostic observation schedule (ADOS) and/or the autism diagnostic interview-revised (ADI-R). Any of the above embodiments may further include, prior to administering the agent, assaying the subject one or more symptoms selected from a low level or activity of BDNF and a low level or activity of TrkB.

Any of the above embodiments may further include, subsequent to administering the agent, assaying in the subject one or more symptoms selected from the group inclusive of intellectual disability, delayed development, sleep disturbance, epilepsy, jerky movements (especially hand-flapping), social-interaction difficulties, communication challenges, repetitive behaviors, ataxia, craniofacial dysmorphology, difficulty with adaptive skills, difficulty standing or walking, inability to walk, ophthalmoplegia, brain atrophy, retinitis pigmentosa, severe limitation of speech and language, easily provoked laughter, a happy demeanor with frequent smiling or spontaneous laughter, impaired ocular movement, and autistic behaviors as measured by the autism diagnostic observation schedule (ADOS) and/or the autism diagnostic interview-revised (ADI-R). Any of the above embodiments may further include, subsequent to administering the agent, assaying in the subject one or more symptoms selected from a low level or activity of BDNF and a low level or activity of TrkB. In such embodiments, the subject may demonstrate improvement in one or more of the symptoms relative to a pre-treatment value.

In any of the above embodiments, the subject may be a newborn or a subject 1 day to 30 years of age. In particular embodiments, the subject may be 1 day to 15 years of age or 1 day to 10 years of age.

In any of the above embodiments, the subject may be a subject that does not have one or more conditions selected from the group inclusive of Alzheimer's disease, Huntington's disease, Parkinson's disease, Rett syndrome, traumatic brain injury, spinal cord injury, age-associated neuronal degeneration, excitotoxicity, stroke, neuropathic pain, depression, obesity, bipolar disorder, aggression, or substance abuse. In any of the above embodiments, the subject may be a subject that does not have one or more conditions selected from the group inclusive of hepatitis C, chronic fatigue syndrome, viral infection, influenza, bacterial infection, middle ear infection, strep throat, pneumonia, typhoid, bronchitis, urinary tract infection, malaria, fungal infection, sinusitis, multiple sclerosis, arrhythmia, ventricular arrhythmia, ventricular tachycardia, ventricular fibrillation, angina, atrial fibrillation, hypertension, metabolic alkalosis, hypochloremia, cancer, osteoporosis, bone lytic diseases, ischemia, silent ischemia, gastric disorders, and osteoclast hyperactivity.

### DEFINITIONS

By “microcephaly associated autism disorder” is meant a range of disorders of neural development characterized by small head size (microcephaly), intellectual disability, such as an intelligence quotient (IQ) of 70 or less, and may include other autistic symptoms. Additional symptoms may include, e.g., epilepsy, loss of speech, craniofacial dysmorphology, ataxia, difficulty with adaptive skills, difficulty walking, inability to walk, ophthalmoplegia, brain atrophy, and retinitis pigmentosa.

By “Christianson syndrome” is meant a neuro-genetic syndrome caused by one or more mutations in the Nhe6 gene and involving one or more symptoms selected from postnatal microcephaly, intellectual disability, epilepsy, non-verbal status, autistic features, craniofacial dysmorphology, ataxia, epilepsy, seizures, difficulty with adaptive skills, difficulty walking, inability to walk, ophthalmoplegia, brain atrophy, and retinitis pigmentosa.

By “Angelman-like syndrome” is meant neuro-genetic syndrome characterized by one or more symptoms selected from intellectual and developmental disability, sleep disturbance, seizures, jerky movements (especially hand-flapping), frequent laughter or smiling, and a happy demeanor.

By “autistic symptoms” is meant one or more of the core symptoms of autism including social-interaction difficulties, communication challenges, and a tendency to engage in repetitive behaviors as measured by the autism diagnostic observation schedule (ADOS) and/or the autism diagnostic interview-revised (ADI-R). Additional autistic symptoms can include epilepsy, seizures, ataxia, loss of speech, and intellectual disability.

By “brain derived neurotrophic factor (BDNF)” is meant a secreted protein that is a member of the “neurotrophin” family of growth factors, which are related to the canonical “nerve growth factor” (NGF). BDNF binds to the TrkB receptors on the surface of cells. BDNF acts on neurons of the central nervous system and the peripheral nervous system, helping to support the survival of existing neurons, and encourage the growth and differentiation of new neurons and synapses. In the brain, it is active in the hippocampus, cortex, and basal forebrain—areas vital to learning, memory, and higher thinking.

By “level or activity” is meant the expression level of a polypeptide (e.g., BDNF or TrkB) in the brain and the associated signal transduction downstream of the polypeptide (e.g., BDNF or TrkB) in the brain. Accordingly, an increase in the activity of a protein may include an increase in the activity of a signaling pathway that includes that protein.

By “tyrosine related kinase B (TrkB)” is meant a type of receptor tyrosine kinase that mediates its actions by causing the addition of phosphate molecules on certain tyrosines of polypeptides in the cell, thus activating cellular signaling. TrkB is the receptor for BDNF, neurotrophin-4, and neurotrophin-5.

By “mimetic” is meant a substance with similar pharmacological effects to another substance, e.g., similar to BDNF. In the present invention, a BDNF mimetic is a substance that can bind to its receptor, e.g., TrkB and produce a biological effect similar to that of BDNF.

By “agonist” is meant a compound that mimics the action of a naturally occurring substance, e.g., BDNF, and can bind to a receptor of a cell and trigger a response by that cell.

By “cell expressing BDNF” is meant a cell (e.g., a mammalian cell) which expresses BDNF. The cell can naturally produce BDNF. Alternatively, the cell can contain a recombinant nucleic acid molecule encoding BDNF, introduced into the cell by transfection, electroporation or viral methods. Electroporation, as used herein, is a method of introducing exogenous nucleic acid molecules into cells by applying an external electric field that causes an increase in the permeability of the cell plasma membrane and uptake of the nucleic acid molecules into the cell. Transfection, as used herein, is a method of introducing exogenous nucleic acid molecules into mammalian cells by chemically opening pores in the cell membrane (e.g., by application of calcium phosphate), to allow uptake of the exogenous nucleic acid molecules. Alternatively, transfection may also be performed by mixing a cationic lipid with the exogenous nucleic acid molecules to produce liposomes that fuse with the cell membrane and deposit the exogenous nucleic acid molecules inside cells.

By “induced pluripotent stem cell (iPSC)” is meant a type of pluripotent stem cell artificially derived from a non-pluripotent cell—typically an adult somatic cell (e.g., skin cell)—by inducing a “forced” expression of specific genes. These stem cells can differentiate into a cell type of choice, for example in the current invention, into specific types of neurons.

By “ataxia” is meant a condition involving a lack of voluntary coordination of muscle movements. Ataxia is a non-specific clinical manifestation implying dysfunction of the parts of the nervous system that coordinate movement, such as the cerebellum.

### BRIEF DESCRIPTION OF THE DRAWINGS

The application file contains drawings executed in color (FIGS. 1-14). Copies of this patent or patent application with color drawings will be provided by the Office upon request and payment of the necessary fee.

FIG. 1 is a set of microscopy images showing that NHE6 localizes to growing axon tracts during development. FIG. 1A shows anti-NHE6 antibodies labeling developing axonal tracts in the embryonic mouse brain. Transverse section of E15.5 brain: NHE6 (green) is found in the cortical plate (CP), striatum (STR) and thalamus (THA) co-localizing with L1 (red), a marker for growing axons. Nuclei are shown in blue. Scale bar, 100 μm. FIG. 1B shows NHE6 labeling major long-range fiber tracts in developing, postnatal mouse brain. In coronal sections, NHE6 (green) co-localizes with L1 (red) in the corpus callosum (CC) (top row), anterior commissure (AC) (second row), the hippocampal formation (HP) (third row), and fimbriae (FB) (bottom row). Nuclei are labeled with Hoechst (blue). Scale bar, CC and AC 50 μm, HP and FB 100 μm.

FIG. 2 is a set of images showing NHE6 protein sub-cellular localization in dissociated hippocampal neurons. FIG. 2A shows immunohistochemistry against NHE6 (red) and GM130 (green), a cis-Golgi marker in 4 days in vitro (DIV) neurons. Scale bar, 5 μm. FIG. 2B shows immunohistochemistry against NHE6 (red) demonstrating co-localization with EEA1 (green), an early endosomal marker in 4 DIV neurons. Arrows show branches of growing neurites. Scale bar, 20 μm. FIG. 2C shows hippocampal neurons at 10 DIV showing that NHE6 (red) is preferentially localized at the branching points (arrows) in phospho-Tau1-positive axons (green). Scale bar, 5 μm. FIG. 2D shows 9 DIV neurons in which anti-NHE6 (red) co-labels phospho-Tau1-positive axons (green) and MAP2-positive dendrites (blue). Scale bar, 10 μm. FIG. 2E shows confocal microscopy images of cultured 21 DIV hippocampal neurons immunolabelled for NHE6 (red), MAP2 (blue) and the presynaptic marker anti-SV2. Inserts are the higher magnification of the boxed regions. Arrows indicate regions of co-localization between NHE6 and SV2 suggesting presynaptic localization of NHE6. Scale bar, 2 μm. FIG. 2F shows confocal images of 21 DIV cultured hippocampal neurons immunolabelled for NHE6 (red), MAP2 (blue) and the postsynaptic marker anti-PSD95. Inserts are the higher magnification of the boxed regions. Scale bar, 2 μm. FIG. 2G shows immunogold electron micrograph of neurites in culture. Arrows show clusters of NHE6 staining presumed to be endosomes along neurite. Scale bar, 500 nm.

FIG. 3 is a set of images showing that NHE6 is required for dendrite and axonal branching. FIG. 3A shows that cultured hippocampal neurons from P0-1 NHE6-null mice show fewer neurite branches compared to wild type littermates at 2 DIV (top panels), and reduced axonal and dendrite branching at 5 DIV (bottom panels). Scale bar, 20 μm. Primary cultures were transfected with GFP-vector in order to label the processes. 5 DIV images show neurolucida traces from reconstructed confocal Z-stacks of GFP-labeled neurons. Cell body is labeled with a red line, axon is labeled with a green line and dendrites are labeled in various colors. Branch points are depicted as dots. FIG. 3B shows quantification of the branching at 2 DIV (WT=5 pups, n=299 cells; MUT=4 pups, n=262 cells, 3 litters) and at 5 DIV (WT=3 pups, n=31 cells; MUT=5 pups, n=40 cells, 3 litters). Error bars are SE; *p<0.05, **p<0.01, ***p<0.001. FIGS. 3C-3D show that NHE6-null mice have reduced branching in vivo in hippocampal CA1 and CA3 regions. FIG. 3C shows images of hippocampal pyramidal neurons analyzed on Golgi-Cox stained coronal brain sections of P21 male mutant (MUT) and wild type (WT) littermates using Neurolucida software. The two panels depict representative inverted micrographs of pyramidal neurons on CA1 (top) and CA3 (bottom) regions. Scale bars, 200 μm. FIG. 3D shows quantification of branching defect in hippocampal neurons is shown as the average mean±SEM of primary dendrites, dendritic branching points, and total number of branches in CA1 pyramidal neurons (WT=4 pups, 105 cells; MUT=5 pups, 77 cells) and CA3 (WT=4 pups, 64 cells; MUT=5 pups, 52 cells) basal and apical dendrites; *p<0.01, **p<0.001, ***p<0.0001, ****p<0.000001. FIGS. 3E-3F show that cortical pyramidal neurons of cortical layer V also show reduced branching in vivo in NHE6-null mice. FIG. 3E shows images of coronal brain sections show in vivo YFP-labeled apical (top) and basal (bottom) dendrites in cortical pyramidal neurons in the mid-anterior region of the cerebral cortex are shown for NHE6-null and wild-type male littermates. Scale bars, 50 μm. FIG. 3F shows quantification of branching defect in cortical pyramidal neurons is shown as the average mean±SEM of primary dendrites, dendritic branching points and total number of branches in the lateral cerebral cortex (WT=2, 69 cells; MUT=2, 72 cells); *p<0.05, **p<0.01, ***p<0.005.

FIG. 4 shows that NHE6 requires the protein cation exchange function to rescue neuronal morphogenesis. FIGS. 4A-4B show overexpression of NHE6 but not an exchanger-defective NHE6 rescues neuronal morphogenesis in NHE6 mutant neurons. FIG. 4A shows P0-1 NHE6 mutant neurons that were transfected after 1 DIV with constructs expressing GFP (top left), NHE6-HA (top right), or HA-tagged exchanger-defective NHE6 (E255Q/D260N) (bottom left). Also, neurons from wild-type littermates were transfected with GFP-expressing constructs alone. Neurons were fixed and imaged at 5 DIV. Representative images show neurolucida traces of reconstructed confocal Z-stacks of GFP-labeled neurons. Inserts show the expression of transfected E255Q/D260N or NHE6-HA (stained with anti-HA antibody) in the soma of neurons and demonstrates equivalent staining and localization of protein, appearing similar to endogenous protein distribution. Scale bar, 20 μm. FIG. 4B shows quantification of axonal and dendrite branching (MUT cells: GFP construct, 3 pups, n=50 cells; E255Q/D260N construct, 3 pups, n=49 cells; NHE6-HA construct, 3 pups, n=46 cells; WT cells: GFP construct, 3 pups, n=26 cells; 3 litters). Data are shown as mean±SE (*p<0.05, ***p<0.001). FIGS. 4C-E show the NHE6 E255Q/D260N exchanger-deficient mutant construct. FIG. 4Ci shows alignments of amino acid or nucleotide sequence of predicted ion transport region of NHE6-HA and E255Q/D260N. Mutated amino acids or nucleotides are unshaded. FIG. 4Cii shows sequence trace of E255Q/D260N. Mutated nucleotides are underlined. FIG. 4D is a western blot showing HeLa cells transfected with HA-tagged NHE6 WT, E255Q/D260N or vector. Lysates were subjected to western blots probed with anti-NHE6 antibody. Equivalent level of protein expression and equivalent bands were observed between mutant NHE6 E255Q/D260N and NHE6 WT construct. FIG. 4E shows hippocampal neurons expressing HA-tagged NHE6 WT or E255Q/D260N that were fixed at 5 DIV and stained with anti-NHE6 and anti-HA antibody. E255Q/D260N showed the same subcellular localization as NHE6 WT construct. These experiments suggest that the point mutations that impair the exchanger domain do not affect protein stability or trafficking. GFP was used to visualize the morphology of hippocampal neurons. Scale bar, 10 μm.

FIG. 5 shows that NHE6 deficient mice have reduced synaptic field potentials for a given stimulus. FIG. 5A shows that extracellular synaptic field potentials in acute hippocampal slices are diminished in the mutants (Nhe6^{-/y}) compared to wild type (Nhe6^{+/y}) littermates. Curves are shown as the mean±SEM of the initial negative slope of the fEPSP as a function of increasing stimulation current (Nhe6^{+/y}, n=12 animals; Nhe6^{-/y}, n=18 animals; p<0.0001. P value was obtained using a two way ANOVA non-matched analysis for the difference between the curves corresponding to each genotype). FIG. 5B shows representative traces at increasing stimulation of slices from mutant (Nhe6^{-/y}) and control (Nhe6^{+/y}) males are shown at 0 μA (black), 100 μA (purple), 200 μA (red). FIG. 5C shows paired-pulse facilitation (PPF) in mutant and wild-type male littermates suggests no defects in pre-synaptic release probability. The average ratios of the initial negative fEPSP slopes after two synaptic stimuli elicited at different time intervals for Nhe6^{-/y} (n=9), i.e. PPF, is indistinguishable from Nhe6^{+/y} (n=17). Ratios are shown as mean±SEM. FIG. 5D shows superimposed representative traces of PPF responses at 50 ms interval for the control (blue) and mutant (red) paired littermates. Scale bar: 1 mV, 10 ms.

FIG. 6 shows NHE6 mutant neurons with reduced synapses, decreased spine density and increased immature spines. FIG. 6A shows cultured hippocampal neurons from NHE6-null mice that show reduced synapses compared to wild-type littermates at 10 DIV, as visualized by SV2 staining (red) (Left images). Neurons were transfected with GFP and immunostained with SV2. Scale bar, 5 μm. Quantification of SV2 densities per 10 μm length in WT and MUT neurons (WT: n=21; MUT: n=24; 3 litters). Error bar, mean±SE; ***p<0.001. FIGS. 6B-6E show in vivo analysis of Golgi stained dendritic spines that shows reduced spine number as well as reduced maturation. FIG. 6B shows high magnification images of both basal (bottom) and apical (top) CA1 pyramidal dendrites in WT and MUT P21 old littermates. Black arrows show immature spines on WT and MUT dendrites. Scale bar, 20 μm. FIG. 6C shows a diagram of spine classification criteria used for spine morphology analysis. Immature spines are composed of (a) filopodia spines, (b) thin spines, and (c) stubby spines; mature spines included (d) mushroom spines, (e) branched spines, and (f) detached spines. FIG. 6D is a histogram showing reduction in total spine density in MUT pyramidal neurons compared to WT pyramidal neurons (WT=3, N=38 cells, n=5292 spines; MUT=4, N=47 cells, n=5546 spines) of CA1 hippocampal region. Average spine number per 10 μm of dendrite length is shown as mean±SEM, **p<0.05. FIG. 6E shows analysis of spine shape in apical and basal dendrites, which showed more immature spines in MUT animals compared to WT littermates. Immature and mature spines are shown as a percentage of total spine number, mean±SEM; *p<0.005, **p<0.0006, ***p<0.00001, ****p<0.0000001; (WT=3, N=38 cells, n=5292 spines; MUT=4, N=47 cells, n=5546 spines).

FIG. 7 shows that ectopic low-pH endosomes in axons and dendrites are observed in the absence of NHE6. FIG. 7A shows hippocampal neurons from NHE6-null and wild type mice that were transfected with GFP constructs at 1 DIV and stained with LysoTracker red at 5 DIV. i-vii are high-magnification images of the boxed regions. Boxes on left reflect cell body. Arrows and numbered boxes on the right indicate LysoTracker (yellow) labeled, low-pH endosomes along the dendrite and axon of hippocampal neurons. Scale bar, 20 μm. FIG. 7B shows the average positions of LysoTracker red puncta in neurites that were measured as a distance from the center of the soma. (WT: 3 pups, n=18 cells; MUT: 3 pups, n=17 cells). Black line indicates mean distance. (***p<0.001).

FIG. 8 shows that branching defects in NHE6 mutant neurons are rescued by BDNF. FIG. 8A shows NHE6 and TrkB proteins co-localized to the same endosomes. Cultured hippocampal neurons were co-transfected with GFP-TrkB and NHE6-HA at 1 DIV and then immunostained with antibodies to NHE6 (red) and HA (blue). Arrows indicated the co-localization of NHE6-HA and GFP-TrkB in the same endosomes. Scale bar, 5 μm. FIG. 8B shows TrkB levels and phospho-Trk induction after BDNF are decreased in NHE6 mutant neurons. Top: representative western blot analysis of total TrkB and phosphorylated Trk protein levels after BDNF administration for different periods in cultured hippocampal neurons from NHE6 mutant and wild type littermates. Tubulin serves as loading control. Bottom: quantitative analysis of the immunoblot bands. Values were normalized to the respective tubulin levels and then were normalized to time 0 min. % control and % induction were the ratio of mutant/WT. (#, single factor ANOVA, p=8.36E-05, n=8; *, single factor ANOVA, p=2.44E-08, n=4). Data represent means+/−SE. FIG. 8C shows that defects in arborization in NHE6 mutant neurons are rescued by exogenous administration of BDNF. Representative images show neurolucida traces of reconstructed confocal Z-stacks of GFP-labeled WT and MUT neurons with or without 50 ng/ml BDNF. Scale bar, 20 μm. FIG. 8D shows quantification of axonal and dendrite branching with or without 50 ng/ml BDNF (MUT cells: without BDNF, 4 pups, n=47 cells; with BDNF, 4 pups, n=44 cells; WT cells: without BDNF, 3 pups, n=46 cells; with BDNF, 3 pups, n=38 cells; 3 litters). Data are shown as mean±SE (*p<0.05).

FIG. 9 shows the characterization of the rabbit anti-NHE6 polyclonal antibody. FIG. 9A is an image of western blot analysis. Vector alone, vectors expressing full length NHE6 protein (isoform NHE6.0) with hemagglutinin tag at the carboxyl terminal (NHE6-HA), NHE6c-terminus only (amino acid 494-669) with or without HA tag (NHE6c-HA or NHE6c) were transiently expressed in NIH/3T3 cells. Expression was analyzed by immunoblotting with rabbit anti-HA antibody (left) or rabbit anti-NHE6 antibody (right). Western blots showed that both the anti-NHE6 antibody and anti-HA antibody recognized the same bands (the lower panel under anti-NHE6 blotting on the right shows longer exposures to resolve the NHE6c-HA band). In extracts from cells transfected with NHE6-expressing constructs, NHE6 is represented by two bands as anticipated from prior literature, one at approximately 70 kDa and a second at approximately 140 kDa. The NHE6 antibody also recognized two bands in mouse brain extract corresponding approximately to the size of endogenous NHE6 in NIH/3T3 cells (seen in vector-only transfected cells). FIG. 9B shows NHE6-GFP over-expressed in HeLa cells and immunocytochemistry performed with rabbit anti-NHE6 antibody. NHE6 immunofluorescence precisely co-localized with GFP fluorescence. Scale bar, 10 μm.

FIG. 10 shows NHE6 expression in E12.5 embryonic murine brain. Coronal sections of E12.5 embryos showed NHE6 (green) colocalized with L1 (red) in the cortical plate (CP), the ventral telencephalon (VTE) and medial ganglionic eminence (MGE). Scale bar: 100 μm.

FIG. 11 shows the time course of expression of NHE6 protein in developing hippocampus in vivo and in vitro. FIG. 11A is a graph showing the results of an in vivo western blot analysis. NHE6 expression (140 kDa-blue, 70 kDa-red) were normalized to tubulin throughout wild-type mouse hippocampal development at stages P0, P7, P14, and P28 (n=3). Error bars represent standard error. FIG. 11B is an image of a western blot showing developmental regulation of NHE6 in primary neurons in vitro. SV2 (synaptic vesicle marker) protein expression served as a measure of neuronal differentiation in hippocampal neurons at different DIV. Tubulin was used as loading control.

FIG. 12 shows Nhe6 targeted gene disruption in mouse. FIG. 12A is a diagrammatic representation of the Nhe6 gene. A mutant allele was generated that replaced exon 6 for the β-galactosidase gene. FIG. 12B is a schematic of the genotyping strategy showing a lower band of 223 bp present only in the wild type littermates and a 478 bp band present only in the mutant. FIG. 12C is western blot analysis data of mutant (mut) and wild type (wt) littermates showing NHE6 protein is completely absent from brain lysates, while the expression of other NHE proteins, endosomal NHE9 and surface membrane NHE1, are not up-regulated. FIG. 12D is histological analysis data using X-gal staining showing expression of β-galactosidase on a heterozygote mouse in coronal sections of the cortical plate and hippocampus in a fashion that mirrors immunohistochemistry of NHE6 protein.

FIG. 13 shows that measures of presynaptic fiber volley are reduced in NHE6 mutant mice, consistent with a reduced number of axonal fibers. FIG. 13A shows analysis of fiber volley amplitude after DNQX addition in hippocampal recordings. Fiber volley curves are smaller in the mutants (Nhe6^{-/y}) compared to wild type (Nhe6^{+/y}) male littermates. Curves are shown as the mean±SEM of the amplitude ratio to the highest value per experiment as a function of increasing stimulation (Nhe6^{+/y}, n=8; Nhe6^{-/y}, n=8); *p<0.001. FIG. 13B shows representative traces after DNQX addition at increasing stimulation of mutant (Nhe6^{-/y}) and control (Nhe6^{+/y}) males at 100 μA (black), 300 μA (red), 600 μA (green), and 900 μA (purple). Scale bars are 1 mV (vertical) by 5 ms (horizontal).

FIG. 14 shows a schematic of the NHE6/TrkB signaling endosome pathway. FIG. 14A depicts the NHE6/TrkB signaling endosome in wild-type neuron. Left: schema of an endosome. Acidification is regulated by the vacuolar-type H⁺-ATPase (V-ATPase) which pumps protons into the endosome lumen, and NHE6 which allows for proton exit via exchange with Nat Right: TrkB endosome signaling (radio-waves) is depicted. With binding of BDNF, TrkB is endocytosed and signals from within endosomes. Endosomes are either recycled or traffic through a series of increasingly acidic endosomal compartments leading to degradation in the lysosome. This signaling process contributes substantially to promoting arborization in developing neurons. FIG. 14B depicts the NHE6/TrkB signaling endosome in the NHE6 null neuron. Left: without NHE6 proton exit from the endosome is impeded resulting in accelerated acidification of the endosome. Right: upon BDNF binding and endocytosis, the endosomal lumen has accelerated acidification which results in increased degradation of TrkB and attenuated TrkB signaling which leads to an impoverishment of axonal and dendritic arbors.

### DETAILED DESCRIPTION OF THE INVENTION

The invention features methods for the treatment of microcephaly associated autism disorders in a subject by administering an agent that increases the level or activity of BDNF or TrkB in the brain of the subject. The microcephaly associated autism disorder can be e.g., Christianson syndrome or Angelman-like syndrome and such disorders can include one or more of the core symptoms of autism, including social-interaction difficulties, communication challenges and a tendency to engage in repetitive behaviors as measured by the autism diagnostic observation schedule (ADOS) and/or the autism diagnostic interview-revise (ADI-R).

Microcephaly is a developmental disorder in which the head of an affected individual is significantly smaller than a reference size. The reference size may be the head size of other individuals of the same sex and age. The reference size may be determined using charts of head size available in the art. Head size may be measured by circumference, e.g., an occipitofrontal circumference (OFC). The head of an individual having microcephaly may have a circumference 2 or more standard deviations below a reference circumference, such as 2, 3, 4, or 5 standard deviations below the reference circumference. The reference circumference may be the mean circumference of the heads of individuals in a population of individuals having the same or similar age and gender as the affected individual. Methods and standards for the diagnosis of microcephaly are known to those skilled in the art.

Microcephaly may be primary microcephaly or secondary microcephaly. Primary microcephaly refers to microcephaly present at birth. Primary microcephaly, in many cases, relates to a brain development defect. Secondary microcephaly refers to the failure of normal head growth after birth, resulting in progression to microcephaly or increasingly severe microcephaly. In some cases, secondary microcephaly may be caused by a condition or disorder that disrupts growth or development.

Angelman syndrome is a neurodevelopmental disorder. In some instances, it is caused by a lack of functional UBE3A. Symptoms of Angelman syndrome include intellectual disability, severe limitation of speech and language, seizures, ataxia, easily provoked laughter, and dysmorphic facial features. Angelman syndrome may be associated with microcephaly. Other symptoms of Angelman syndrome are known in the art. Methods for the diagnosis of Angelman syndrome are also known in the art.

Angelman-like syndrome is a condition resulting in phenotypes similar to those observed in connection with Angelman syndrome. Symptoms include intellectual disability, ataxia, severe limitations in language and speech, epilepsy, and a happy demeanor with frequent smiling or spontaneous laughter. Angelman-like syndrome may be associated with microcephaly. Other symptoms of Angelman-like syndrome are known in the art. Methods for the diagnosis of Angelman-like syndrome are also known in the art.

Christianson syndrome is an X-linked Angelman-like syndrome caused by mutation of Nhe6 (solute carrier family 9, isoform 6). Symptoms of Christianson syndrome include delayed development or developmental regression, intellectual disability, an inability to speak, impaired ocular movement, ataxia, and difficulty standing or walking. Affected children often have a happy demeanor, demonstrating frequent smiling and spontaneous laughter. Christianson syndrome may be associated with microcephaly. Other symptoms of Christianson syndrome are known in the art. Methods for the diagnosis of Christianson syndrome are also known in the art.

A subject having or diagnosed with a microcephaly associated autism disorder may be a fetus, a child, or an adult. The subject may be, e.g., a newborn. The subject may be 1 year old or less, e.g., 1 day, 10 days, 20, days, 1 month, 2 months, 3 months, 6 months, 9 months, or 12 months old. The subject may also be more than 1 year old, e.g., 2, 3, 4, 5, 10, 15, 20, 25, 30, 40, 50, 60, 70, 80, 90, 100, or more years old. The methods of the present invention may be used to treat a subject of any of these ages having or diagnosed with a microcephaly associated autism disorder, although, in some instances, a microcephaly associated autism disorder may result in premature death, e.g., death prior to age 30.

### Microcephaly Associated Autism Disorder Caused by Mutation in Nhe6 Gene

Mutations in the X-linked, endosomal Na⁺/H⁺ Exchanger 6 (Nhe6, also known as SLC9A6) gene cause a neuro-genetic syndrome involving postnatal microcephaly and autistic features. Nhe6 is among the most commonly mutated genes causing X-linked developmental brain disorders. The first reports of mutations in Nhe6 were associated with an Angelman-like syndrome (AS). One of the pedigrees determined to have a Nhe6 mutation was a large South African pedigree previously reported by Christianson et al. (J. Med. Genet.; 36: 759-766. incorporated herein by reference).

Christianson syndrome (CS) is now the commonly used term for the condition associated with Nhe6 mutations. Published mutations in the highly related endosomal gene Nhe9 are also implicated in autism with epilepsy. Recently, Nhe9 has been shown to be significantly upregulated and Nhe6 to be downregulated in postmortem brains from patients with idiopathic autism.

Axonal and dendritic growth and arborization have been implicated as a potential mechanism in microcephaly associated autism disorders as both occur at greater rates in disorders such as Christianson syndrome.

### Nhe6 and Endosomal Signaling in Axonal and Dendritic Branching

Within the developing brain, strict control of axon and dendritic branching is critical for circuit development and function. This process may be influenced by a variety of signaling pathways amenable to environmental and pharmacologic intervention, including endosomal signaling via the BDNF/TrkB pathway. The endocytic machinery has an important role in governing neuronal arborization via functions including controlling receptor trafficking, recycling and degradation, and modulating signaling pathways essential for neurite growth and arborization. For example, the role of the endosomal pathway in neuronal morphogenesis is exemplified by the discovery of the Drosophila shrub mutations that demonstrate ectopic dendritic and axonal branching due to loss of a coiled-coil protein homologous to the yeast protein Snf7, a key component in the ESCRT-III (endosomal sorting complex required for transport) complex that is essential for endosomal to lysosomal sorting (Sweeney et al., 2006).

The endosomal compartment is divided into component parts with increasingly acidic luminal environment, specifically early endosome (pH˜6.3), recycling endosome (pH˜6.5), late endosome (pH˜5.5), and lysosome (pH˜4.7). The vacuolar H⁺-ATPase (V-ATPase) is a pump that mediates acidification of endosomes and lysosomes. The endosomal Na⁺/H⁺ exchangers (NHEs) allow movement of cations down their concentration gradients (Na⁺ and/or K⁺ in, and H⁺ out), and counters the V-ATPase by regulating relative alkalization of the lumen as well as endosomal size. The gradation of intra-luminal acidity likely serves a number of critical functions, yet this has also been scarcely investigated in developing neurons. It was demonstrated in differentiating sympathetic neurons that a discontinuous gradient of endosome acidification along the axon with more high-pH endosomes, namely pH˜6.6 to 8.2, near growth cones or axonal branch points, and more low-pH endosomes, pH 4.2 to 6.0 proximally near the soma exists. The effect of pH on neurotrophin signaling has been studied in a single study in PC12 cells wherein NGF and NT3 binding and activation of TrkA are decreased by increasing lumen acidity. The pH of endosomal compartments may contribute to signaling via the BDNF/TrkB pathway. In particular, acidification of endosomal compartments may attenuate signaling via the BDNF/TrkB pathway.

NHE proteins have broad importance in cell biology, and in particular in neurology, given the spontaneous mutation in Nhe1 in the slow-wave epilepsy mouse, and the various anti-epileptic medicines that alter regulation of proton concentrations. Ten NHE genes are known in vertebrates. The structure of NHE proteins generally involves a twelve-membrane spanning motif that harbors the Na⁺ and H⁺ exchange activity and is highly conserved across family members, and a large, less conserved carboxyl domain that is thought to involve protein interaction and regulation. In addition to showing distinct gene expression patterns across tissues, the NHE proteins also show distinct subcellular distributions. NHE1-5 are localized to the cell surface and NHE6-9 are organellar. NHE6 and NHE9 are the “endosomal NHEs” and appear to be localized to early recycling and late endosomes respectively, in non-neuronal cell lines.

NHE6-associated endosomes are enriched within growing axons and dendrites and at branchpoints. NHE6-null neurons display an expansion of the low-pH endosomal compartment within axons and dendrites, and drastically impoverished axonal and dendritic arborization. Consistent with this observation, NHE6-null mice have impaired circuit function. Furthermore NHE6-null neurons demonstrate attenuated TrkB signaling, indicating that endosomal signaling through BDNF/TrkB may be perturbed in microcephaly associated autism disorders such as CS, which have impoverished axonal and dendritic arborization. Thus administering agents that increase the level or activity of BDNF and/or TrkB in the brain may be a plausible approach for treatment of one or more symptoms of microcephaly associated autism disorders, e.g., those caused by mutations in Nhe6.

Method of Treatment for Microcephaly Associated Autism Disorders Using Agents that Increases the Level or Activity of BDNF and/or TrkB in the Brain

The invention features methods for treatment of subjects (e.g., humans) suffering from a microcephaly associated autism disorder (e.g., CS or AS), by administering an agent that increases the level or activity of BDNF, or TrkB, or both in the brain of the subject.

BDNF binds to the TrkB receptors on the surface of cells and acts on neurons of the central nervous system and the peripheral nervous system, helping to support the survival of existing neurons, and encourage the growth and differentiation of new neurons and synapses.

The agents that can be used in the methods of the invention to increase the level or activity of BDNF in the brain can be any one or more of BDNF (Genbank Gene ID: 627), a BDNF agonist, a BDNF mimetic, a TrkB agonist, a cell expressing recombinant BDNF, a BDNF encoding recombinant nucleic acid molecule encapsidated within a recombinant virus, and an agent that decreases the acidity of endosomes.

### BDNF and BDNF Mimetics for Use in the Methods of the Invention as BDNF Agonists

The agent that can be used in the methods of the invention can be naturally occurring purified BDNF. Alternatively, the agent can be a recombinant form of BDNF produced using recombinant protein expression and purification methods known in the art.

The agent that can be used in the methods of the invention can also be a BDNF mimetic that can bind to the TrkB receptor. The BDNF mimetic can be a non-peptide mimetic as described in US 20120004310 and US 20070060526 (each of which is incorporated herein by reference). The BDNF mimetic can be a small molecule such as those described in Massa et al. (J. Clin. Invest. 2010; 120: 1774-1785, incorporated herein by reference) and in Schmid et al. (J. Neurosci. 2012; 32(5): 1803-1810, incorporated herein by reference). These BDNF mimetics can include LM22A-2, LM22A-3, and LM22A-4.

The BDNF mimetic can be a peptide mimetic e.g., a tricyclic dimeric peptide such as the one described in O'Leary and Hughes (J. Biol. Chem. 2003; 278: 25738-25744, incorporated herein by reference) that may mimic loop 2 of BDNF, a dipeptide BDNF mimetic, e.g., as described in US 20110312895 (incorporated herein by reference) that may mimic loop 4 of BDNF, or a tripeptide mimetic e.g., as described in Massa et al (e.g., LM22A-1) that may mimic loop 2 of BDNF. The peptide mimetic can also be a cyclic pentapeptide (e.g., cyclic pentapeptide 2) that may be designed to mimic a cationic tripeptide sequence in loop 4 of BDNF as described in Fletcher et al. (J. Biol. Chem. 2008; 283: 33375-33383, incorporated herein by reference). Typically “loop mimetics” mimic loops 2 or 4 of BDNF since these are the regions of BDNF that are involved in TrkB binding.

The BDNF mimetic can also be a chimeric neurotrophic factor as described in U.S. Pat. No. 5,488,099 (incorporated herein by reference). Small molecules that can be used as BDNF mimetics, in the methods of the invention, by functioning as TrkB ligands, can be selected from those described in Xie and Longo (Prog. Brain Res. 2000; 128: 333-47, incorporated herein by reference). The BDNF mimetic can be deoxygedunin as described in Jang et al. (PLoS One 2010; 5(7): e11528, incorporated herein by reference). The agent that can be used with the methods of the invention can be any one of the TrkB activating antibodies as described in Qian et al. (J. Neurosci. 2006; 26(37): 9394-9403, incorporated herein by reference).

The agent that can be used with the methods of the invention for increasing the level or activity of BDNF/TrkB signalling can also be 7, 8-dihydroxyflavone as described in Jang et al. (Proc. Natl. Acad. Sci. 2010; 107(6): 2687-2692, incorporated herein by reference).

The agent that can be used with the methods of the invention for increasing the level or activity of BDNF/TrkB signalling can also be 4′-dimethylamino-7,8-dihydroxyflavone as described in Liu et al. (J. Med. Chem. 2010; 53 (23): 8274-8286, incorporated herein by reference).

The agent that can be used with the methods of the invention for increasing the level or activity of BDNF/TrkB signalling can also be neurotrophin 4 or neurotrophin 5, which are ligands for TrkB, as described in Gao et al. (J. Neurosci. 1995; 15(4): 2656-2667, incorporated herein by reference).

Additional BDNF agonists are described in U.S. Pat. No. 7,763,462 (incorporated herein by reference).

### Use of Cells and Viruses Expressing a Recombinant BDNF in the Methods of the Invention

The agent that can be used in the methods of the invention can also be a cell expressing a recombinant BDNF, e.g., an iPSC that can express natural and/or recombinant BDNF and is applied to the brain such that the cell secretes the BDNF and increases BDNF/TrkB signaling in the brain. The iPSC can be engineered to express BDNF by introduction of a recombinant BDNF gene cloned into a recombinant expression vector. The recombinant expression vector including the recombinant BDNF gene can be introduced into the iPSCs by transfection, electroporation or viral methods. Electroporation can include introducing exogenous nucleic acid molecules into cells by applying an external electric field that causes an increase in the permeability of the cell plasma membrane and uptake of the nucleic acid molecules into the cell. Transfection includes introducing exogenous nucleic acid molecules into mammalian cells by chemically opening pores in the cell membrane (e.g., by application of calcium phosphate), to allow uptake of the exogenous nucleic acid molecules. Alternatively, transfection may also be performed by mixing a cationic lipid with the exogenous nucleic acid molecules to produce liposomes that fuse with the cell membrane and deposit the exogenous nucleic acid molecules inside cells.

The iPSC can be artificially derived from a non-pluripotent cell—typically an adult somatic cell (e.g., skin cell)—by inducing a “forced” expression of specific genes. These stem cells can differentiate into a cell type of choice, for example in the current invention, into specific types of neurons.

The agent that can be used in the methods of the invention can also be a BDNF encoding recombinant nucleic acid molecule encapsidated within a recombinant virus. In this case, the recombinant BDNF gene is cloned into a recombinant viral expression vector.

Construction of vectors for recombinant expression of BDNF for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory Press, NY, 4^{th} Ed. (2012). For generation of efficient expression vectors, it is necessary to have regulatory sequences that control the expression of the recombinant BDNF gene. These regulatory sequences include promoter and enhancer sequences and are influenced by specific cellular factors that interact with these sequences.

Promoter and enhancer regions have been described in the art. Methods for maintaining and increasing expression of transgenes, e.g., recombinant BDNF, in quiescent cells include the use of promoters including collagen type I (1 and 2), SV40, and LTR promoters. The promoter can be a constitutive promoter selected from the group inclusive of: ubiquitin promoter, CMV promoter, JeT promoter (e.g., as described in U.S. Pat. No. 6,555,674, incorporated herein by reference), SV40 promoter, Elongation Factor 1 alpha promoter (EF1-alpha), RSV, Mo-MLV-LTR. Examples of inducible/repressible promoters include: Tet-On, Tet-Off, Rapamycin-inducible promoter, Mx1. The promoter can also be a constitutive or inducible promoters known in the art. Further expression enhancing sequences include but are not limited to Woodchuck hepatitis virus post-transcriptional regulation element, WPRE, SP163, CMV enhancer, and Chicken β-globin insulator or other insulators.

Transgene expression, e.g., recombinant BDNF expression, may also be increased for long term stable expression using cytokines to modulate promoter activity. Several cytokines have been reported to modulate the expression of transgene from collagen 2 (1) and LTR promoters. For example, transforming growth factor (TGF), interleukin (IL)-1, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll (E)) may also be used to increase transgene, e.g., recombinant BDNF, expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll (E) promoter-enhancer, and reduces transgene expression.

The expression vector may further include sequences such as a sequence coding for the Cre-recombinase protein, and LoxP sequences. A way of ensuring transient expression of the recombinant BDNF is through the use of the Cre-LoxP system which results in the excision of part of the inserted DNA sequence either upon administration of Cre-recombinase to the cells or by incorporating a gene coding for the recombinase into the virus construct. Incorporating a gene for the recombinase in the virus construct together with the LoxP sites and a structural gene (a recombinant BDNF gene in the present case) often results in expression of the structural gene for a period of approximately five days or more.

The expression vector containing the recombinant nucleic acid encoding BDNF can be encapsidated within a recombinant virus e.g., recombinant adeno-associated virus (AAV), recombinant retrovirus, recombinant lentivirus, recombinant poxvirus, recombinant rabies virus, recombinant pseudo-rabies virus, and recombinant herpes simplex virus, papovavirus, human immunideficiency virus (HIV), and adenovirus. These viruses are then applied to the subject (e.g., a patient) so that the endothelial cells can be infected by these viruses and the BDNF can then be expressed in endothelial cells.

Preferred viruses include lentiviruses and adeno-associated viruses (AAVs). Both types of viruses can integrate into the genome without cell divisions, and both types have been tested in pre-clinical animal studies. Methods for preparation of AAVs are described in the art e.g., in U.S. Pat. No. 5,677,158, U.S. Pat. No. 6,309,634, and U.S. Pat. No. 6,683,058, each of which is incorporated herein by reference. Methods for preparation and in vivo administration of lentiviruses are described in US 20020037281 (incorporated herein by reference). Preferably, a lentivirus vector is a replication-defective lentivirus particle. Such a lentivirus particle can be produced from a lentiviral vector including a 5′ lentiviral LTR, a tRNA binding site, a packaging signal, a promoter operably linked to a polynucleotide signal encoding the recombinant protein, an origin of second strand DNA synthesis and a 3′ lentiviral LTR.

Retroviruses are most commonly used in human clinical trials, since they carry 7-8 kb and since they have the ability to infect cells and have their genetic material stably integrated into the host cell with high efficiency (see, e.g., WO 95/30761; WO 95/24929, each of which is incorporated herein by reference). Oncovirinae require at least one round of target cell proliferation for transfer and integration of exogenous nucleic acid sequences into the patient.

For use in human patients, the retrovirus must be replication defective. This prevents further generation of infectious retroviral particles in the target tissue. Instead the replication defective virus becomes a “captive” transgene stable incorporated into the target cell genome. Typically in replication defective vectors, the gag, env, and pol genes have been deleted (along with most of the rest of the viral genome). Heterologous DNA (in case of the present invention, the recombinant nucleic acid molecule encoding BDNF) is inserted in place of the deleted viral genes. The heterologous genes may be under the control of the endogenous heterologous promoter, another heterologous promoter active in the target cell, or the retroviral 5′ LTR (the viral LTR is active in diverse tissues).

The viruses can be introduced into the body by intravascular injection. For localized targeting, virus injection from an IV catheter has already been used to achieve spatially discrete expression (e.g., of a single chamber of the heart or localized cerebral vasculature). In cases where the desired target can be accessed by catheterization, local transduction would then provide spatial specificity to BBB opening. Alternatively, direct intra-cerebral virus injection can be used to target specific vessels. While more invasive than catheterization, this procedure is less invasive than implantation of a deep-brain stimulator and does not require maintenance of hardware in the brain. Further, in cases where more elaborate surgery is already standard—tumor removal, epilepsy surgery—local transduction could be achieved. Alternatively, direct peripheral virus injection can be used to target specific vessels outside of the central nervous system.

Viruses encoding recombinant BDNF may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Additional agents that can be used in the methods of the invention to increase the level or activity of BDNF and/or TrkB can be selected from the ones described in US 20080139472 (incorporated herein by reference), such as, one or more agents selected from the group inclusive of an anti-depressant drug, an anti-anxiolytic drug, an anti-psychotic drug, an acetylcholinesterase inhibitor, a delta- or mu-opioid receptor agonist, epidermal growth factor (EGF), nerve growth factor (NGF) and/or a bicyclic or tricyclic antidepressant and/or a selective serotonin reuptake inhibitor (SSRI) and/or an antidepressant selected from the group inclusive of fluoxetine, desipramine, 2-methyl-6-(phenylethynyl)-pyridine), and venlafaxine and/or an anxiolytic agent (e.g., afobazole, buspirone, lorazepam, diazepam, fluoxetine, eszopiclone, paroxetine, sertraline, citalopram, clomipramine, clonazepram, St. John's wort, etc.) and/or an anti-psychotic (e.g., quetiapine, chlorpromazine, fluphenazine, perphenazine, prochlorperazine, thioridazine, trifluoperazine, mesoridazine, promazine, triflupromazine, levomepromazine, chlorprothixene, flupenthixol, thiothixene, zuclopenthixol, haloperidol, droperidol, pimozide, melperone, clozapine, olanzapine, risperidone, quetiapine, ziprasidone, amisulpride, paliperidone, cannabidiol, LY2140023, etc.) and/or a histone deacetylase inhibitor (e.g., sodium butyrate, sodium phenylbutyrate, sodium phenylacetate, pivaloyloxymethylbutyrate, pyroxamide, depsipeptide, oxamflatin, benzamide derivative MS-275, trichostatin A, suberoylanilide hydroxamic acid, trapoxin A, trapoxin B, Cyl-1, Cyl-2, HC-toxin, WF-3161, chlamydocin, apicidin, MS-275 (previously called MS-27-275), depudecin, etc.) and/or an acetylcholinesterase inhibitor (e.g. huperzine A, physostigmine, pyridostigmine, ambenonium, demarcarium, edrophonium, neostigmine, tacrine (tetrahydroaminoacridine), donepezil (a.k.a. E2020), rivastigmine, metrifonate, galantamine, phenothiazine, etc.) and/or a neuropeptide whose expression is regulated by cocaine or other amphetamine, and/or cystamine or nictotine, and/or estrogen or adrenocorticotropin, and/or dopamine, norepinephrine, L-DOPA, serotonin, or analogues thereof, and/or Semax.

The agent that can be used in the methods of the invention to increase the level or activity of BDNF in the brain of the subject can also be a modulator of alpha-amino-3-hydroxy-5-methyl-isox-azole-4-propionic acid (AMPA) type glutamate receptors as described in US 20080139472.

Use of Agents that Reduce the Acidity of Endosomal Compartments

The pH of endosomal compartments may modulate the level or activity of BDNF, TrkB, or both. An agent that increases the level or activity of BDNF, TrkB, or both, may be an agent that reduces the acidity of endosomal compartments, e.g., in cells of the brain. An agent that reduces the acidity of endosomal compartments may be selected from amantadine (FDA approved; previously marketed in the United States as Symadine® and Symmetrel®), amiodarone (FDA-approved; marketed as Cordarone®, Pacerone®, and Nexterone®; used to treat life-threatening arrhythmias (e.g., ventricular tachycardia, ventricular fibrillation) in patients who have already taken other anti-arrhythmic medicines), ammonium chloride, azithromycin, bafilomycin A1, benzolactone enamides (e.g., salicylihalamide, lobatamide, apicularen, oximidine, and cruentaren), bepridil (previously approved for clinical use as a calcium channel blocker to treat angina), diphyllin (a natural compound isolated from Cleistanthus collinus), indolyls (e.g., indole derivatives of bafilomycin, such as INDOL0), macrolactones (e.g., archazolid and azithromycin), monensin, nigericin, plecomacrolides (e.g., bafilomycin A1 and concanamycin), quinolines (e.g., amodiaquine, chloroquine, and hydroxychloroquine), or sulfonamides (e.g., 16D2 (5-bromo-2-{[(4-chloro-3-nitrophenyl)sulfonyl]amino}-N-(2,5-dichlorophenyl)benzamide) and 16D10 (5-chloro-2-{[(4-chloro-3-nitrophenyl)sulfonyl]amino}-N-(4-chlorophenyl)benzamide), identified in a chemical genetic screen for inhibitors of membrane trafficking; see Nieland et al. Traffic 5(7): 478-492 (2004), which is herein incorporated by reference). In particular embodiments, the agent that reduces the acidity of endosomal compartments in cells of the brain is a quinoline. In still more particular embodiments, the agent that reduces the acidity of endosomal compartments in cells of the brain is chloroquine. Any of the above-mentioned agents may be used alone or in combination with one or more additional agents, optionally including a second agent selected from those provided herein. Without wishing to be limited to any particular mechanism of action or effect, the aforementioned agents may function in the manner provided in Table 1. Other agents capable of reducing the acidity of endosomal compartments are known in the art.

TABLE 1

| Agent | Mechanism | Effect |
|---|---|---|
| Amantadine | Viral proton | Reduces vesicular |
| | pump inhibitor; | acidification (i.e., |
| | Diffusable | increases pH); Antiviral |
| | weak base; | (Influenza A); Antidyskinetic |
| | NMDA receptor | (Parkinson's Disease); |
| | antagonist | Potentially increases |
| | | consciousness or awareness |
| | | (Traumatic Brain Injury); |
| | | Decreases toxic effects of |
| | | glutamatergic neurotransmitter |
| | | system |
| Amiodarone | Non-selective | Mildly increases membrane- |
| | calcium | proximal endosomal pH; |
| | channel blocker | Inhibits β-secretase |
| | | and γ-secretase |
| | | cleavage of amyloid |
| | | precursor protein |
| Ammonium chloride | Diffusable | Becomes protonated and |
| | weak base | increases organelle pH |
| Azithromycin | V-ATPase | Reduces endosomal/lysosomal |
| | inhibitor | acidification |
| | | (i.e., increase pH) |
| Bafilomycin A1 | V-ATPase | Reduces endosomal/lysosomal |
| | inhibitor | acidification |
| | | (i.e., increase pH) |
| Benzolactone | Class of | Reduces endosomal/lysosomal |
| enamides | v-ATPase | acidification |
| | inhibitors | (i.e., increase pH) |
| Bepridil | Non-selective | Mildly increases membrane- |
| | calcium | proximal endosomal pH; |
| | channel blocker | Inhibits β-secretase |
| | | and γ-secretase |
| | | cleavage of amyloid |
| | | precursor protein |
| Diphyllin | v-ATPase | Reduces endosomal/lysosomal |
| | inhibitor | acidification |
| | | (i.e., increases pH); |
| | | Antiviral activity |
| Indolyls | Class of | Reduces endosomal/lysosomal |
| | v-ATPase | acidification |
| | inhibitors | (i.e., increases pH) |
| Macrolactones | Class of | Reduces endosomal/lysosomal |
| | v-ATPase | acidification |
| | inhibitors | (i.e., increase pH) |
| Monensin | Carboxylic | Reduces acidification of |
| | ionophore that | endosomes |
| | exchanges | (i.e., increases pH) |
| | protons for K⁺ | |
| | and Na⁺ | |
| Nigericin | Carboxylic | Reduces acidification of |
| | ionophore that | endosomes |
| | exchanges | (i.e., increases pH) |
| | protons for K⁺ | |
| | and Na⁺ | |
| Plecomacrolides | Class of | Reduces endosomal/lysosomal |
| | v-ATPase | acidification |
| | inhibitors | (i.e., increase pH) |
| Quinolines | Diffusable | Becomes protonated and |
| (e.g., chloroquine, | weak bases | increase organelle pH |
| hydroxychloroquine) | | |
| Sulfonamides | Inhibitors of | Reduces endosomal/lysosomal |
| (16D2, 16D10) | v-ATPase | acidification |
| | ATPase activity; | (i.e., increase pH) |
| | Proton ionophores | |

Symptoms of Microcephaly Associated Autism Disorders that can be Treated by the Methods of the Invention

Patients suffering from microcephaly associated autistic disorders such as Christianson syndrome or Angelman-like syndrome may have symptoms that include intellectual disability, such as an intelligence quotient (IQ) of 70 or less (e.g., an IQ of 70, 60, 50, 40, 30, 20, 10, or less), epilepsy, loss of speech, craniofacial dysmorphology, ataxia, difficulty with adaptive skills, difficulty walking, inability to walk, ophthalmoplegia, brain atrophy, autistic symptoms, and/or retinitis pigmentosa.

The agents that can be used in the methods of the invention can be used to treat, ameliorate, reverse or slow the progression of any one or more of the above symptoms or other symptoms of microcephaly associated autistic disorders known in the art. For example, an agent of the present invention may increase the IQ of the subject, thereby treating or ameliorating the aspect intellectual disability measured by IQ. The agent may treat or reverse the loss of speech. In some instances, the agents may stop the progressive loss of speech. In certain instances, the agent may increase adaptive skills, fully or partially restoring the ability of a subject to function in normal age appropriate environments. The agent may also help treat or ameliorate epileptic seizures. The agents may also treat, ameliorate, reverse or slow the progression of brain atrophy and/or any neuro-anatomical pathologies symptomatic of microcephaly associated autistic disorders.

### Diagnosis Prior to Treatment

Diagnostic tests to determine whether a patient has a microcephaly associated autistic disorder, such as CS or AS, can be performed prior to administering the agent that increases the level or activity of BDNF or TrkB in the brain of the subject. These tests can include measuring head circumference to determine whether the subject has microcephaly. The microcephaly may be primary or secondary microcephaly.

Diagnostic tests performed prior to treatment can also include measuring the level or activity of BDNF, TrkB, or both in the subject. In some instances, the subject is diagnosed prior to treatment as having a low level or activity of BDNF, TrkB, or both relative to a control subject or reference value. The level or activity of BDNF, TrkB, or both may be determined in cells of one or more tissues, e.g., the brain. The level or activity of BDNF and/or TrkB, e.g., in the brain of the subject, can be less than that of the control subject or reference value by 20% or more (e.g., by 20%, 25%, 30%, 35%, 40%, 50%, 60%, 70%, 80%, or 90% or more). A level or activity of BDNF, TrkB, or both that is less than that of the control subject or reference value by 20% or more may be indicative of a microcephaly associated autistic disorder. The level or activity of BDNF and/or TrkB can be measured by western blot assay, ELISA, or any assay known in the art for measuring BDNF and/or TrkB level and/or activity.

In some instances, the subject is diagnosed as having Angelman-like syndrome prior to treatment. In some instances, the subject is diagnosed as having Christianson syndrome prior to treatment. For instance, the subject may be diagnosed as having one or more mutations in the Nhe6 gene relative to a control subject or reference sequence. A diagnostic test performed prior to treatment can be a genetic test, e.g., a test for the presence of one or more mutations in the Nhe6 gene in the subject relative to a control subject. For example, a mutant Nhe6 gene may encode a NHE6 protein that has mutant amino acids E255Q and/or D260N. The presence of one or more mutations can be detected by any one or more of a genomic sequencing assay, polymerase chain reaction assay, fluorescence in situ hybridization assay, or an immunoassay. In the above mentioned tests, the control subject is a subject without a microcephaly associated autism disorder or any other form of neuro-developmental or psychiatric disorder.

In some instances, the subject is diagnosed with an autism disorder prior to treatment based on one or more symptoms selected from intellectual disability, delayed development, sleep disturbance, epilepsy, jerky movements (especially hand-flapping), social-interaction difficulties, difficulty communicating, severe limitation of speech and language, repetitive behaviors, ataxia, craniofacial dysmorphology, difficulty with adaptive skills, difficulty standing or walking, inability to walk, impaired ocular movement, ophthalmoplegia, brain atrophy, retinitis pigmentosa, easily provoked laughter, a happy demeanor with frequent smiling or spontaneous laughter, or autistic behaviors as measured by the autism diagnostic observation schedule (ADOS) and/or the autism diagnostic interview-revised (ADI-R). Assays for each of these symptoms are known in the art and may be applied to a subject to determine whether the subject is in need of treatment with an agent capable of increasing the level or activity of BDNF and/or TrkB. Assays for these phenotypes may be, e.g., qualitative, quantitative, or a combination thereof.

One or more assays for any of the above symptoms may be applied to a subject prior to treatment at any age at which the symptom may be meaningfully measured. One of skill in the art will know the minimum age at which each symptom may be assayed. For instance, microcephaly may be assayed in a fetus, a newborn, or later. Other symptoms, such as difficulty walking or speaking, may only be meaningfully assayed in subjects having attained an age at which such traits may be manifested in a reference population. In general, a subject to be treated by a method of the present invention may be a fetus, a child, or an adult. The subject may be, e.g., a newborn. The subject may be 1 year old or less, e.g., 1 day, 10 days, 20, days, 1 month, 2 months, 3 months, 6 months, 9 months, or 12 months old. The subject may also be more than 1 year old, e.g., 2, 3, 4, 5, 10, 15, 20, 25, 30, 40, 50, 60, 70, 80, 90, or 100, or more years old.

In certain embodiments of the present invention, the treated subject does not have or has not been diagnosed with one or more, or any, of Alzheimer's disease, Huntington's disease, Parkinson's disease, Rett syndrome, traumatic brain injury, spinal cord injury, age-associated neuronal degeneration, excitotoxicity, stroke, neuropathic pain, depression, obesity, bipolar disorder, aggression, or substance abuse. In certain embodiments, the treated subject does not have or has not been diagnosed with one or more of hepatitis C, chronic fatigue syndrome, viral infection, influenza, bacterial infection, middle ear infection, strep throat, pneumonia, typhoid, bronchitis, urinary tract infection, malaria, fungal infection, sinusitis, multiple sclerosis, arrhythmia, ventricular arrhythmia, ventricular tachycardia, ventricular fibrillation, angina, atrial fibrillation, hypertension, metabolic alkalosis, hypochloremia, cancer, osteoporosis, bone lytic diseases, ischemia, silent ischemia, gastric disorders, or osteoclast hyperactivity,

### Post-Treatment Monitoring

The methods of the invention can further include monitoring the effects of the agents that are administered to the subject. The monitoring can include measuring changes in head circumference, change in speech, change in adaptive skills, change in ability to walk, change in ataxia, change in seizures, and change in IQ as a result of administration of the agents of the method. The change can be an increase or decrease relative to the pre-treatment condition.

In particular embodiments, analysis during or after treatment according to a method of the present invention may demonstrate an increase in the level or activity of BDNF, TrkB, or both in one or more tissues, e.g., in the brain. In some instances, the treated subject has been diagnosed with Christianson syndrome and demonstrates improvement in one or more symptoms of Christianson syndrome. In some instances, the treated subject has been diagnosed with Angelman-like syndrome and demonstrates improvement in one or more symptoms of Angelman-like syndrome. In some instances, the subject has been diagnosed with an autism disorder based on phenotypic information and demonstrates improvement of a relevant phenotype. For instance, a subject having been treated by a method of the present invention may be tested for and demonstrate improvement in or normalization of one or more of intellectual ability, development, sleep patterns or behaviors, epilepsy, jerky movements (especially hand-flapping), social interaction abilities, communication abilities, repetitive behaviors, ataxia, adaptive skills, ability to stand, mobility, ability to walk, ophthalmoplegia, brain atrophy, retinitis pigmentosa, speech and language abilities, ocular movement, or autistic behaviors as measured by the autism diagnostic observation schedule (ADOS) and/or the autism diagnostic interview-revised (ADI-R). In some instances, the subject demonstrates a decrease in or normalization of one or more of the ease with which laughter may be provoked, happiness of demeanor, frequency of smiling, or frequency of spontaneous laughter. In some instances, a subject having been treated by a method of the present invention may be tested for and demonstrate an increase in or normalization of the level or activity of BDNF, TrkB, or both in cells of one or more tissues, e.g., the brain. Assays for each of these symptoms are known in the art and may be applied to a subject to determine whether treatment according to a method of the present invention modulated, improved, or normalized one or more of these symptoms. Assays for these symptoms may be, e.g., qualitative, quantitative, or a combination thereof. In particular embodiments, a symptom is measured both prior to and/or during and/or subsequent to treatment. In such embodiments, diagnostic tests may be used to determine whether treatment with an agent capable of increasing the level or activity of BDNF and/or TrkB has modulated, improved, or normalized the symptoms.

### EXAMPLES

### Experimental Procedures

### Reagents and Antibodies:

LysoTracker Red DND-99 dye was purchased from Molecular probes. FD Rapid GolgiStain kit was purchased from FD Neuro Technologies. Rabbit polyclonal anti-NHE6 antibody was prepared against isoform-specific sequences (aa636-655) of the C terminus of NHE6.0. Antisera were collected and affinity-purified. Mouse monoclonal anti-L1 antibody (clone 324) and chicken anti-MAP2 antibody were purchased from Millipore. Mouse monoclonal anti-GM130 was purchased from BD Transduction Laboratories. Mouse monoclonal anti-EEA1 antibody (G-4) was purchased from Santa Cruz Biotechnology. Mouse anti-phospho-tau1 antibody (clone PHF-1) was kindly provided by Dr. Peter Davies, Albert Einstein College of Medicine.

### Mice:

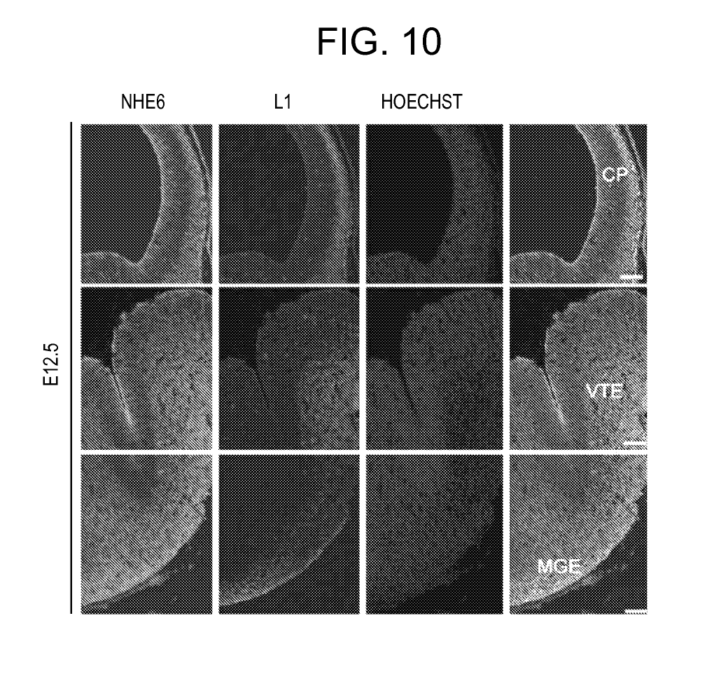

The Nhe6 knockout mice were obtained from Jackson Laboratories. A LacZ-Neo cassette was inserted into exon 6 to inactivate the Nhe6 gene (FIG. 10A). Hemizygous Nhe6−/X females were bred with wild-type Nhe6+/Y or homozygous Nhe6−/Y males. Animals were genotyped by PCR with forward primers (5′ GGGTGGGATTAGATAAATGCCTGCTCT-3′ and 5′-AACAGCTGTGGAGGGATATGTGCT-3′) and reverse primer (5′-AGCTGGCTTTGCGCATGGAGCATT-3′) for wild type (223 bp) and for mutant (478 bp) bands (FIG. 10B). We also performed western blots to confirm that NHE6 protein was absent from the mutant brain lysate (FIG. 10C). All experiments involving mice were carried out in accordance with the US National Institutes of Health Guide for the Care and Use of Animals under the protocols approved by the Brown University Institutional Animal Care and Use Committee.

### DNA Constructs:

Human Nhe6 gene (NM_006359) was cloned into a pReceiver-M07 mammalian expression vector to generate HA-tagged pNHE6-HA. Nhe6 mutant E255Q/D260N was constructed with the QuickChange site-directed mutagenesis kit (Stratagene) according to the manufacturer's protocol with overlapping primers (5′-TCT ATG CAC TTC TTT TTG GTC AAA GTG TCC TCA ATA ATG CTG TTG CCA TAG TGC TGT C-3′) and (5′-GAC AGC ACT ATG GCA ACA GCA TTA TTG AGG ACA CTT TGA CCA AAA AGA AGT GCA TAG A-3′) and was based on prior publication.

### Primary Neuron Culture and Transfection:

Hippocampi were dissected from P0-P1 mice, dissociated with papain (20 units/ml) in Earle's Balanced Salt Solution (EBSS) with bicarbonate at 37° C. for 30 min and triturated with a 1 ml pipette. Hippocampal neurons were placed on 8-well chamber slides coated with 1 mg/ml poly-D-lysine at a cell density of 3×10⁵ cells/ml. For neuronal morphology, neurons at 1 DIV were transfected with EGFP constructs or together with testing constructs at a ratio of 1:1 using Lipofectamine 2000 (Invitrogen) according to manufacturer guidelines. For LysoTracker experiments, GFP transfected neurons at 5 DIV were incubated with 100 nM Lysotracker Red DND-99 at 37° C. for 30 min. After washing with PBS for 3 times, cells were imaged live or cells were fixed with 4% paraformaldehyde for 15 min and Z-series images were obtained under a Zeiss LSM710 confocal laser scanning microscope.

### Immunohistochemistry:

Embryonic or newborn wildtype brains were fixed with 4% paraformaldehyde overnight at 4° C. Brains were then cryoprotected in sucrose by sequentially increasing the sucrose percentage from 10% to 30%. Brains were allowed to sink in sucrose overnight, and were subsequently embedded in OCT freezing media and stored at −80° C. Coronal brain sections of 10 to 14 μm thickness were obtained using a Leica Cryostat. Sections were allowed to dry for at least 30 minutes and were stored at −80° C. Immunohistochemistry was conducted by post-fixing sections for 2 min in 4% paraformaldehyde, rinsing in 1×PBS three times and blocking for 1 hr in 2% goat serum, 1% BSA, 0.1% triton in 1×PBS. Primary antibodies were diluted in blocking solution and incubated for 3 hrs at room temperature or overnight at 4° C. Unbound primary antibody was removed by 5 washes in blocking solution, followed by 5 washes in 1×PBS. Secondary antibody was diluted in blocking solution at 1:1000 and incubated for 1 hr at room temperature. Unbound secondary antibodies were rinsed as above and mounted in Fluoromont-G (Southern Biotech) for viewing under the microscope. Primary cultures of hippocampal neurons were fixed with 4% paraformaldehyde, rinsed with 1×PBS, permeabalized with 0.25% triton X-100, 1×PBS and blocked with 10% goat serum, 1×PBS and 0.1% triton X-100. Primary and secondary antibody incubations were subsequently performed in 2% goat serum, 1×PBS and 0.1% triton X-100. After washing, cultures were preserved by mounting coverslips on slides with Fluoromount-G.

### Immunogold Electron Microscopy Sample Preparation:

Primary cultures of hippocampal neurons were prepared as described above. Cells were fixed with 4% paraformaldehyde with 0.5% glutaraldehyde in 0.15M NaCacodylate buffer for 1 h at 4° C. Samples were rinsed with 0.15M NaCacod buffer twice and quenched with 0.15M NaCacod buffer with 50 mM glycine for 10 min. After having rinsed with 1×PBS, samples continued with antibody labeling and embedding based on standard protocol.

### Golgi-Cox Labelling and Analysis:

P21 day old male mice both mutant and control littermates were perfused with 4% paraformaldehyde without post-fixing. Brains were then processed using the FD Rapid Golgi stain kit. After 2 weeks and 2 days the Golgi processed brains were flash frozen for 1 minute in isopentane pre-cooled in dry-ice and either mounted on specimen disc on OCT without embedding or embedded in TFM (TBS, Durham). Sagittal and coronal brain sections of 150 μm were obtained using a LEICA Cryostat. Sections were further stained with FD Rapid Golgistain kit, dehydrated in sequentially increasing ethanol percentages and cleared three times in Xylene, and mounted using Permount. Z-stack images were obtained of 0.4 to 1 μm stack thickness with a Zeiss microscope driven by Axiovision software.

### Morphometric Analyses:

Images of cultured hippocampal neurons (2 DIV) were captured from randomly chosen fields on a Zeiss LSM710 confocal laser scanning microscope, blinded to genotypes, and traced using Metamorph Neurite Outgrowth software. The number of processes (i.e., neurites emanating directly from the cell body) per cell, the total number and total cumulative length of all neurite branches per cell and the length of the longest neurite per cell were determined. Z-series images of individual GFP-filled neurons at 5 DIV were obtained on a Carl Zeiss 710 confocal microscope and traced manually using Neurolucida software. Quantification of the branch number and total length of axons and dendrites per cell was done on images acquired with 20× objective. Quantitative analysis for LysoTracker red labeled endosomes was performed by using Neurolucida in images acquired with 63× objective. LysoTracker red puncta that completely overlapped with GFP-labeled dendrites or axons were scored as co-localized endosomes. Each experiment was performed on at least three different batches of cultured neurons.

### Electrophysiology:

All experiments were conducted blinded to the genotype. Wild-type and mutant males between P14 to P21 days after birth were used to obtain 400 μm thick coronal brain slices. In most experiments only 1 slice was used per animal per experiment, if multiple slices were used the average of those experiments was used so that the final number represents the number of animals used. fEPSPs were recorded from CA1 pyramidal cells in the stratum radiatum using recording and conditions known in the art. Experiments were analyzed using Labview software. For all slices a 10 minute baseline of stable synaptic responses was obtained before starting the experiment. For input output experiments, stimulus intensity was used as a percentage of 1 mAmp in 10% increments from 0% to a 100%. For paired pulse ratio experiments the stimulus intensity was kept constant and the time intervals between the first and second pulse were changed from 10 ms to 1000 ms. The average of 4 consecutive responses was used for each stimulus intensity point (input output curves) or for each time interval (paired pulse facilitation).

### Statistical Analysis:

Most studies were tested for significance with a Student's t-test for two-group comparisons or one-way ANOVA followed by t-test for multiple comparisons among more than two groups. Two-way ANOVA for non-matched samples for comparison of electrophysiological data. A p-value of <0.05 was considered significant. Data were expressed as mean+SEM.

### Example 1

### Nhe6-Associated Endosomes are Localized to Growing Axons and Dendrites

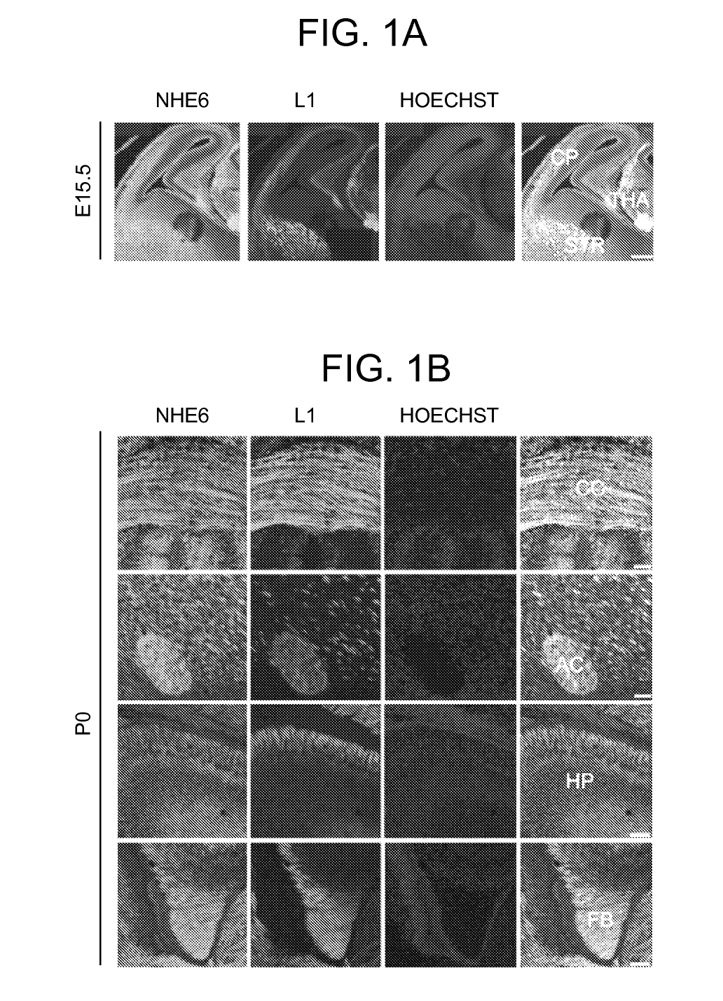

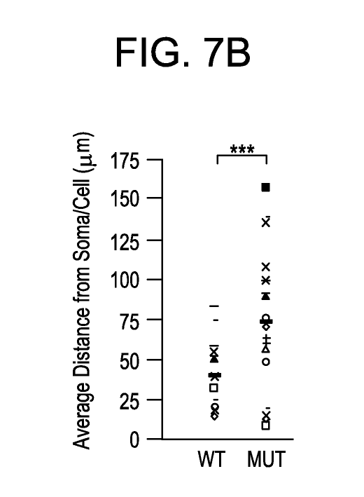

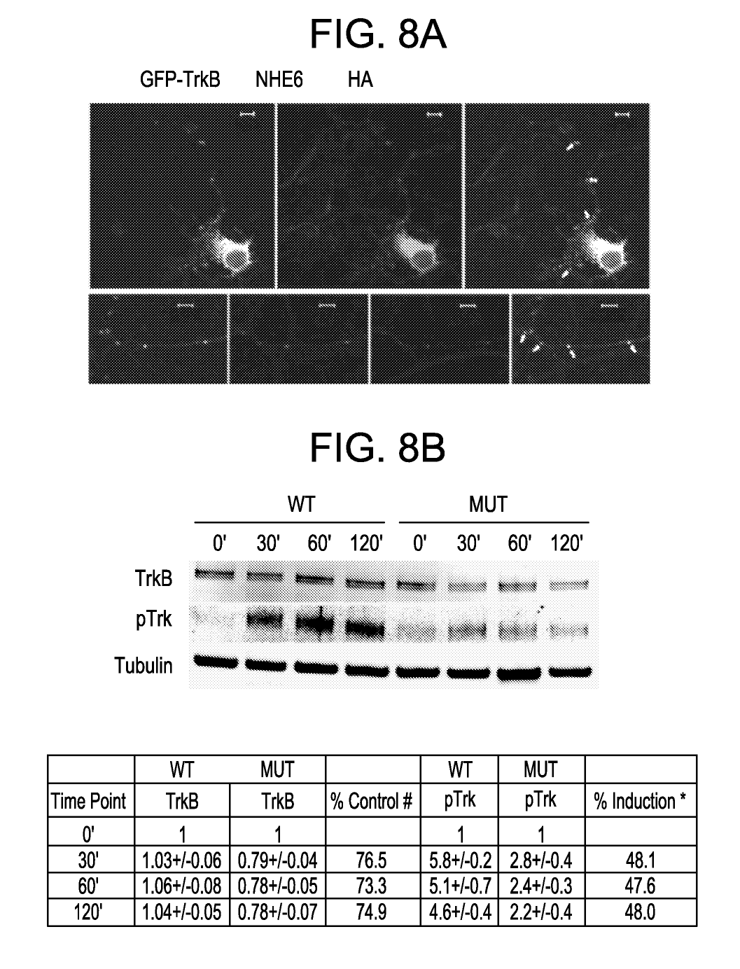

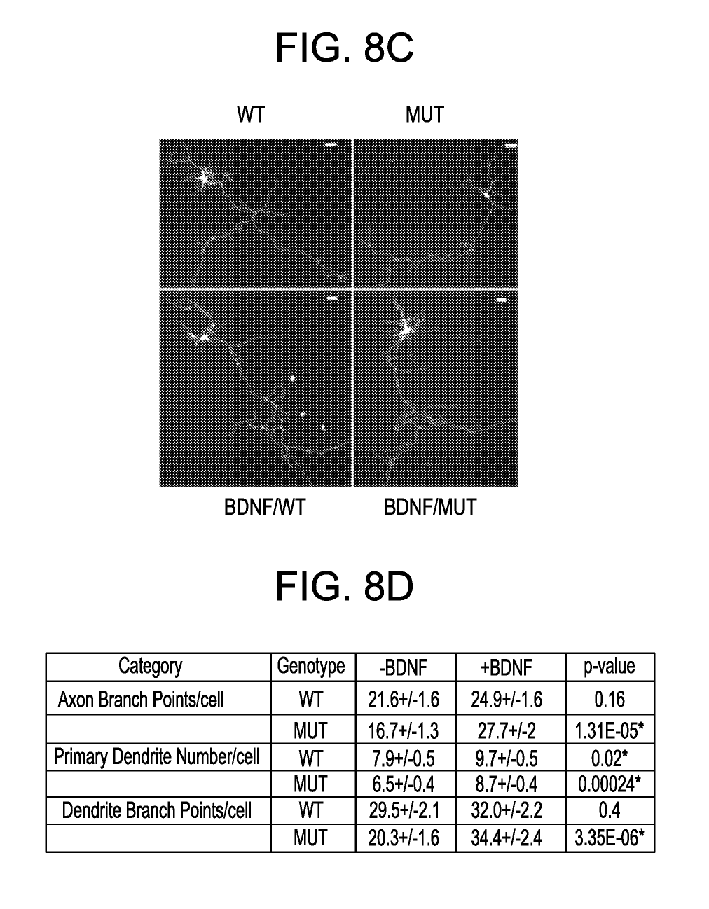

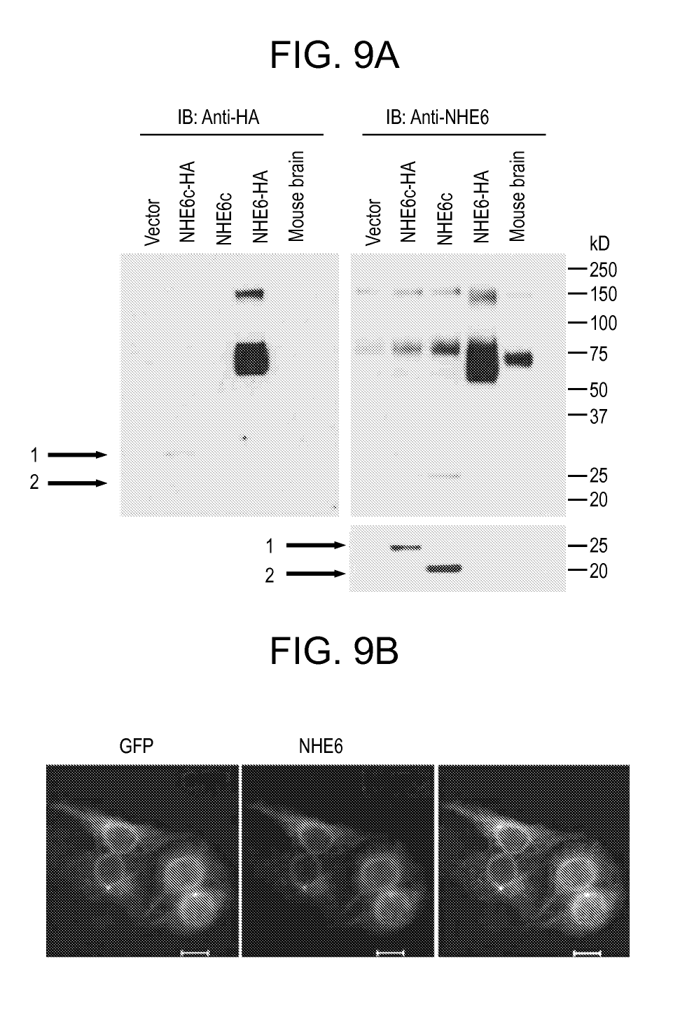

To examine NHE6 function in neuronal development, we generated antibodies against NHE6. (The specificity of our antibody was verified by western blot and immunocytochemistry in FIG. 8.) Immunostaining of NHE6 in developing mouse brain demonstrated a striking and strong staining in growing axon tracts in vivo. NHE6 protein is prominently enriched in growing axonal tracts during development including in cortical plate, striatum and thalamus at embryonic day 15.5 (E15.5), and in major, long-range fiber tracts such as corpus callosum, anterior commissure, hippocampal formation and fimbrae at postnatal day 0 (P0) (FIG. 1). Staining is less prominent in embryonic stages prior to axon development (FIG. 9).

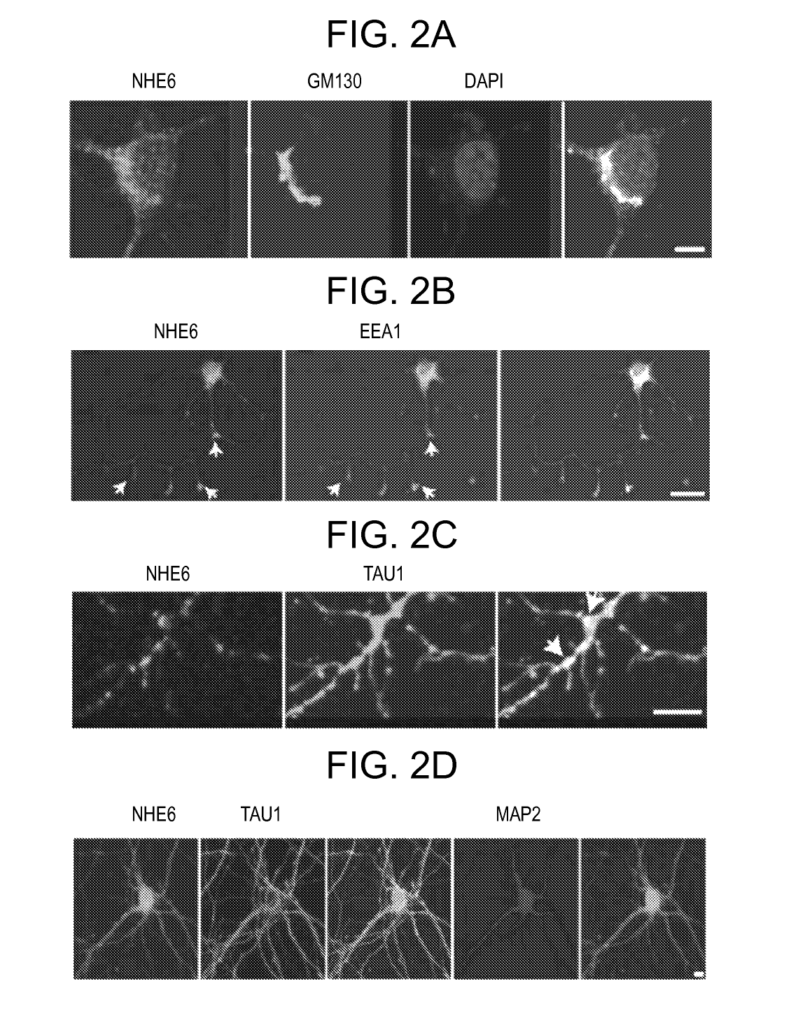

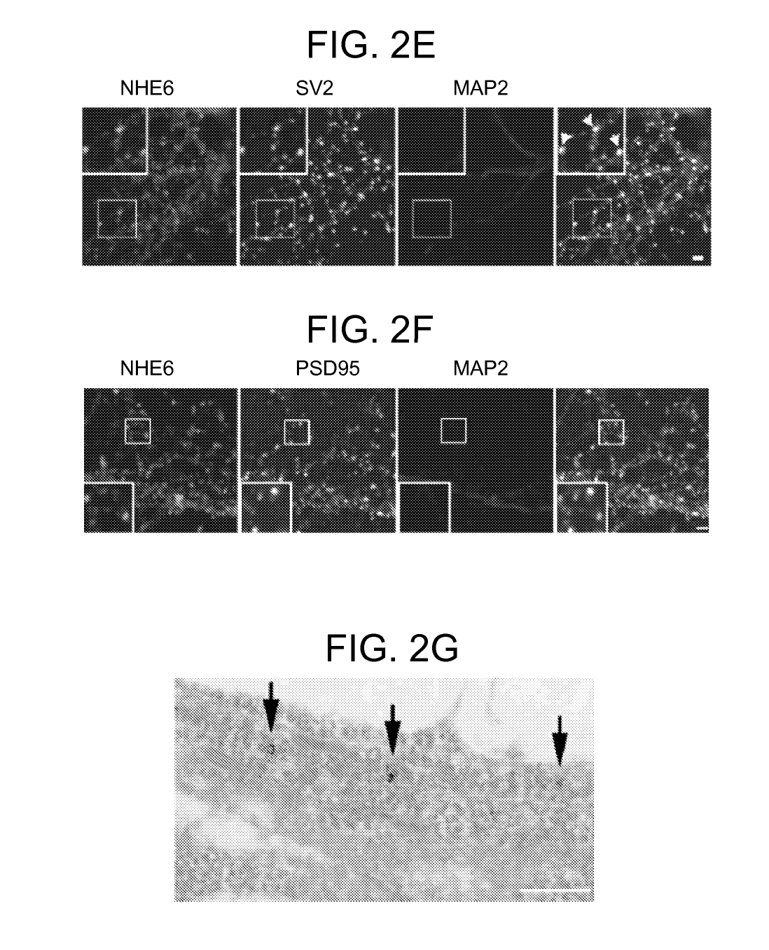

We also examined the subcellular localization of NHE6 in dissociated neurons in vitro. In dissociated hippocampal neurons, NHE6 staining appeared punctate with clusters localized to the perinuclear region, and also prominently within growing axons and also in growing dendrites (FIG. 2). Although NHE6 punctae were notable in both axons and dendrites, there was a relatively greater prominence to the staining in axons over dendrites. The perinuclear staining appeared adjacent to anti-GM130 staining (a cis-Golgi marker), suggesting NHE6 localization adjacent to Golgi apparatus (FIG. 2A). NHE6 also co-localized with markers of early endosomes, such as EEA1 in soma and neurites (FIG. 2B). Co-localization of NHE6 and EEA1 was seen in the perinuclear region, the branch points and the tips of neurites (FIG. 2B arrows, with anti-EEA1 staining at branch points and tips of growing dendrites; FIG. 2C arrows, with anti-Tau1 staining at branch points in growing axons). Using immuno-electron microscopy on dissociated neuronal cultures, NHE6 staining appeared in discrete clusters within growing neurites (FIG. 2G).

In older cultures, we continue to observe NHE6-positive punctae enriched in axons and dendrites (FIG. 2D); however, in adult mouse brain, NHE6 staining is considerable less strong in mature axon tracts (data not shown), suggesting an active role of NHE6 endosomes in axon and dendrite development. Overall, our data were consistent with expression of NHE6 in early and recycling endosomes, with most heavy staining in growing axons, and some staining in dendrites. Localization within growing neurites was frequently observed at branch points. As synapses appear in the culture, we observed NHE6-positive punctae frequently co-localized to SV2-positive punctae, a pre-synaptive marker (FIG. 2E) and rarely overlapping with the post-synaptic marker PSD95 (FIG. 2F) suggesting that NHE6 localizes to a subset of synapses, with an apparently greater frequency at the pre-synapse.

### Example 2

### Loss of Nhe6 Leads to Impoverished Axon and Dendrite Branching In Vitro and In Vivo

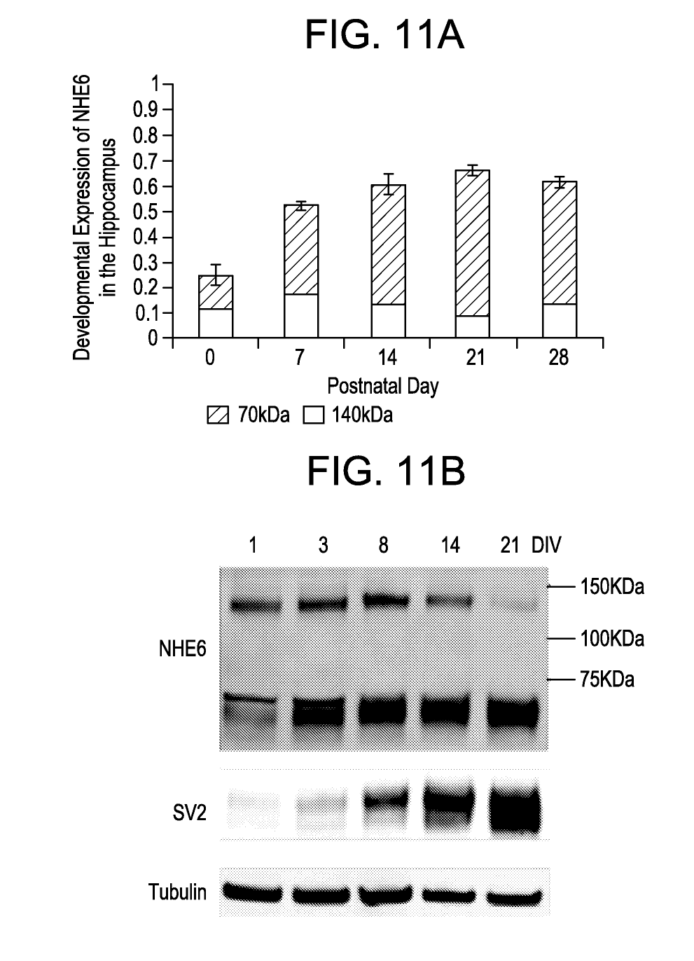

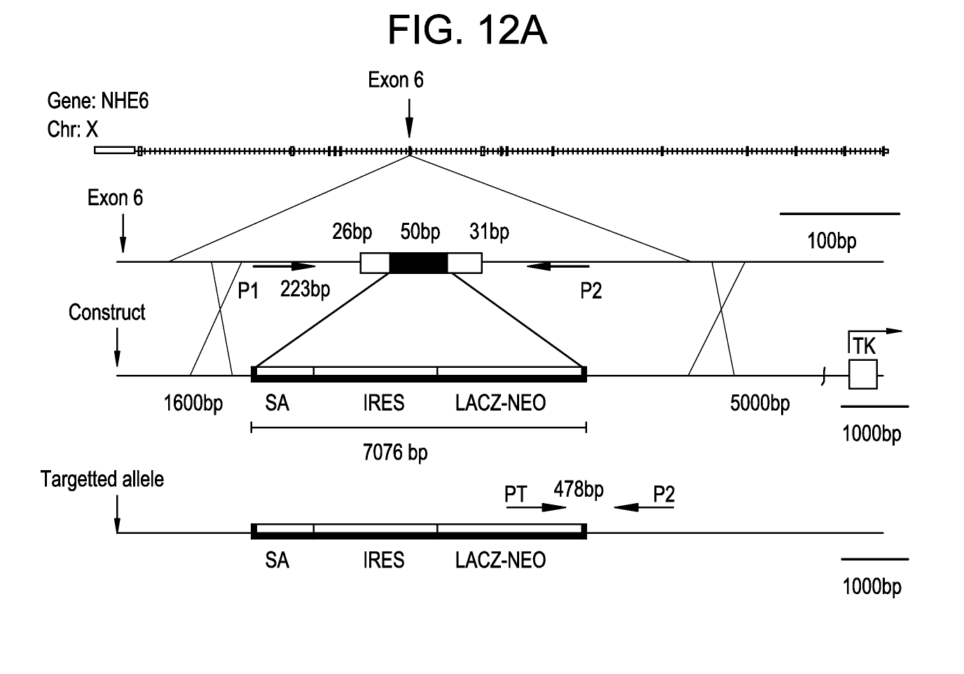

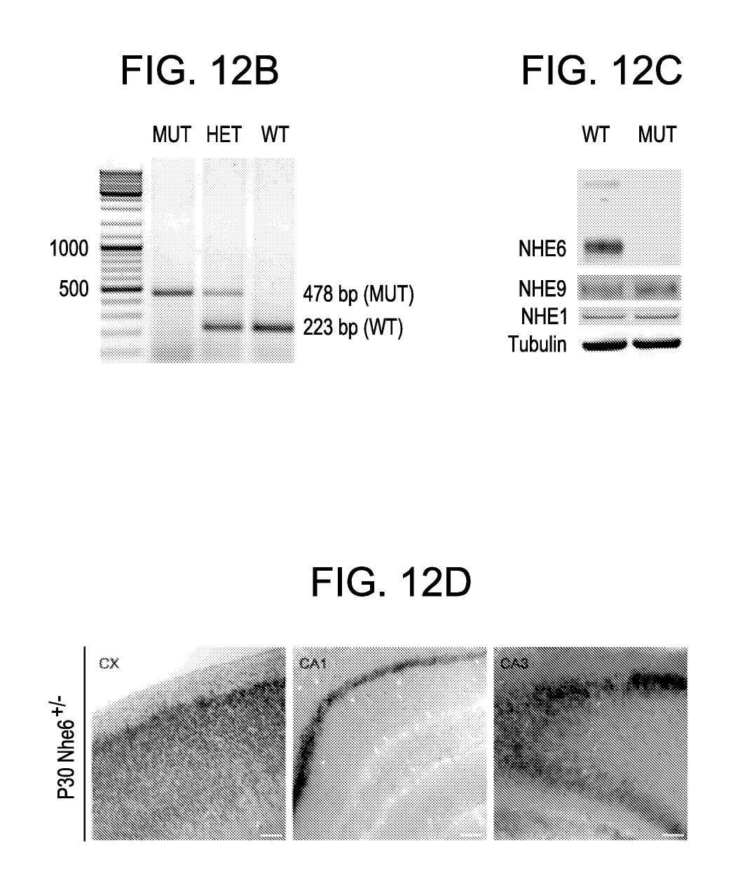

Given the striking staining in growing fiber tracts in vivo and the prominent localization at branch points of growing neurites, we hypothesized that NHE6-associated endosomes play a role in neurite outgrowth and branching. Quantitative western blot analysis of proteins extracted from hippocampal neurons on progressive days in vitro (DIV) and in vivo, revealed that NHE6 expression increases significantly when neurite growth accelerates, suggesting a role for NHE6 in neurite morphogenesis (FIG. 11). To discern NHE6 function, we established an NHE6-null mouse line in which a LacZ-Neo cassette was inserted into exon 6 to inactivate the Nhe6 gene (FIG. 12). We tested the mutant line by western blot and demonstrated the absence of the anticipated bands specific to NHE6. Interestingly, NHE9 and NHE1, other disease-associated NHE genes, are not upregulated in the absence of NHE6. NHE6-null mice showed an inconsistent increase in unexplained mortality in the first month (approximately 10-20% of pups). Otherwise mutant mice do not generally appear distinct from their wild-type littermates.

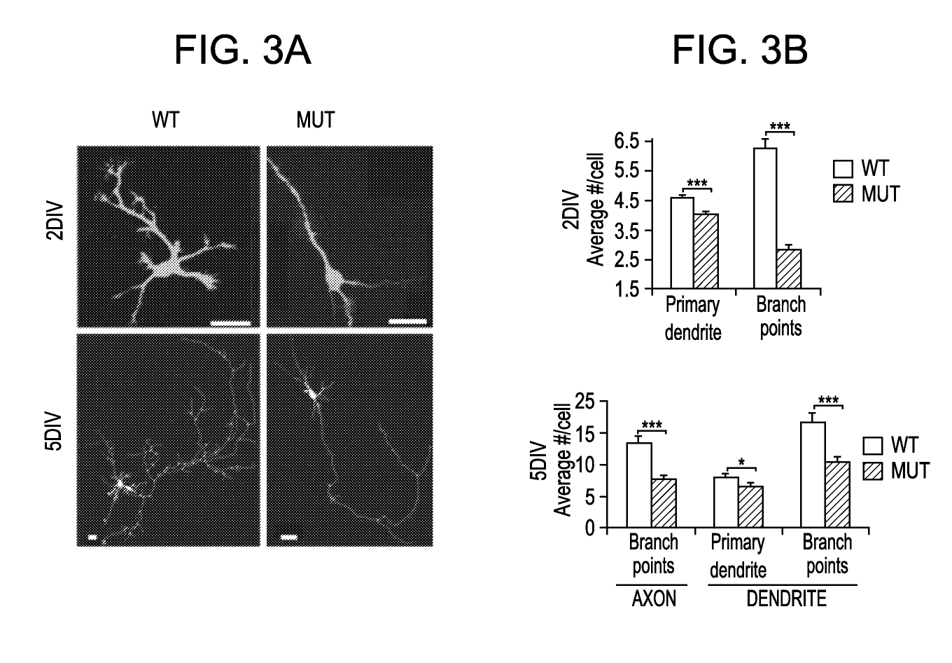

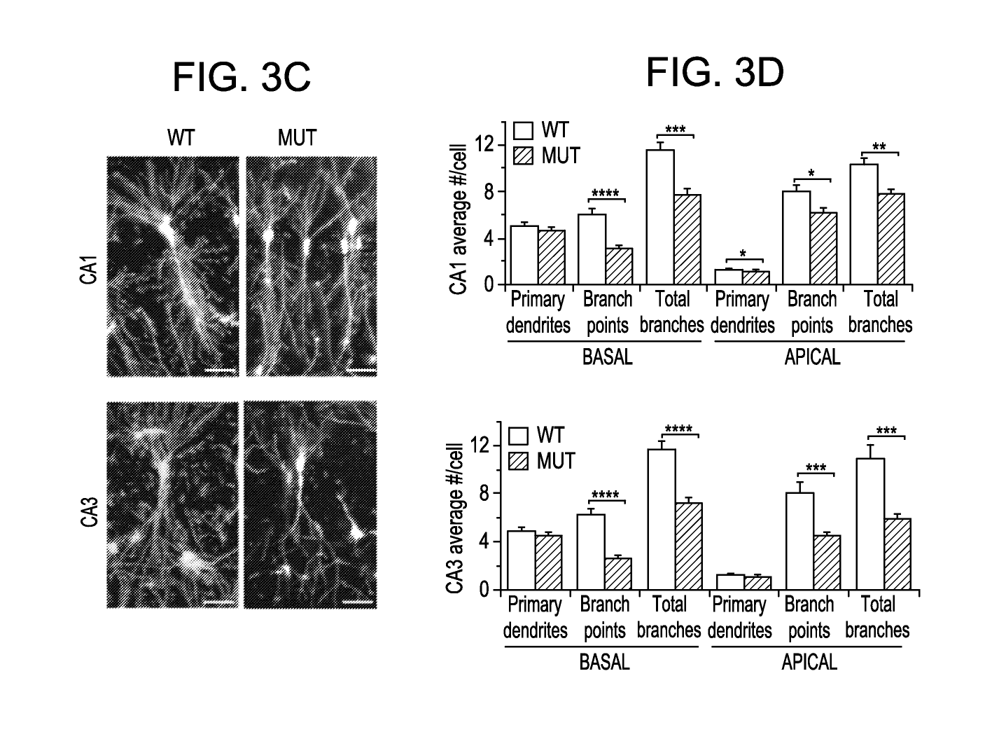

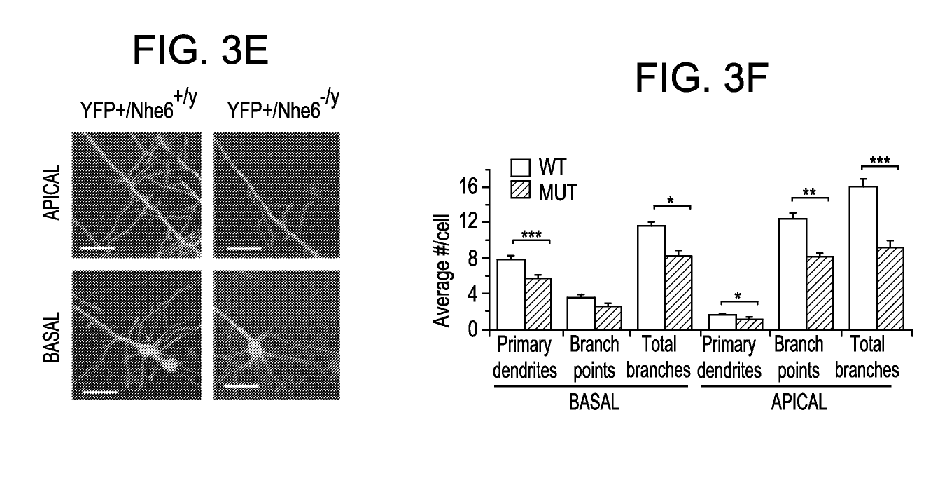

Mouse hippocampal cultures from postnatal day 0-1 NHE6-null and wild-type littermates were prepared, transfected with GFP-expressing vectors, and assayed at 2 DIV and 5 DIV for neuronal morphogenesis. As determined by computer tracing analysis (FIGS. 3A and 3B), hippocampal neurons from NHE6-null mice showed a significant reduction in axon branch points. Dendritic branch points and primary dendrite numbers were also reduced. Because these observations were made as early as 2 DIV, we hypothesize that these morphogenesis defects were primary failures in neuronal differentiation as opposed to secondary effects of over-pruning due to failures in synapses. Synapses are not substantially present prior to 5 DIV (FIG. 11B). The observation that there are defects in primary dendrite numbers and not simply branches also supports this interpretation.

We next examined neuronal morphogenesis in vivo in both hippocampus and cortex. Abnormalities in neuronal morphogenesis that were observed in hippocampal neurons in vitro were corroborated by quantification of Golgi-Cox stained dendritic arbors in vivo (FIGS. 3C and 3D). Dendritic branching points and total number of branches in both CA3 and CA1 hippocampal pyramidal neurons in vivo were significantly reduced in NHE6-null mice compared to littermate controls at postnatal day 21 in basal and apical dendrites (FIGS. 3C and 3D).

NHE6-null mice expressing YFP from a Thy-1 promoter were used to analyze further the branching defect in cortex in the absence of NHE6. Layer V cortical pyramidal neurons were analyzed from the lateral cortex, as single cells could be easily traced. Similar to our findings with Golgi-Cox stained brains, we found YFP-labeled neurons in NHE6-null mice had reduced apical and basal branching points, and reduced total number of branches and number of primary dendrites compared to controls (FIGS. 3E and 3F).

### Example 3

### Defects in Neuronal Morphogenesis are Rescued by Cell-Autonomous Expression of Wild-Type Nhe6 in Mutant Neurons, but not by a Cation-Exchange Deficient Form of Nhe6

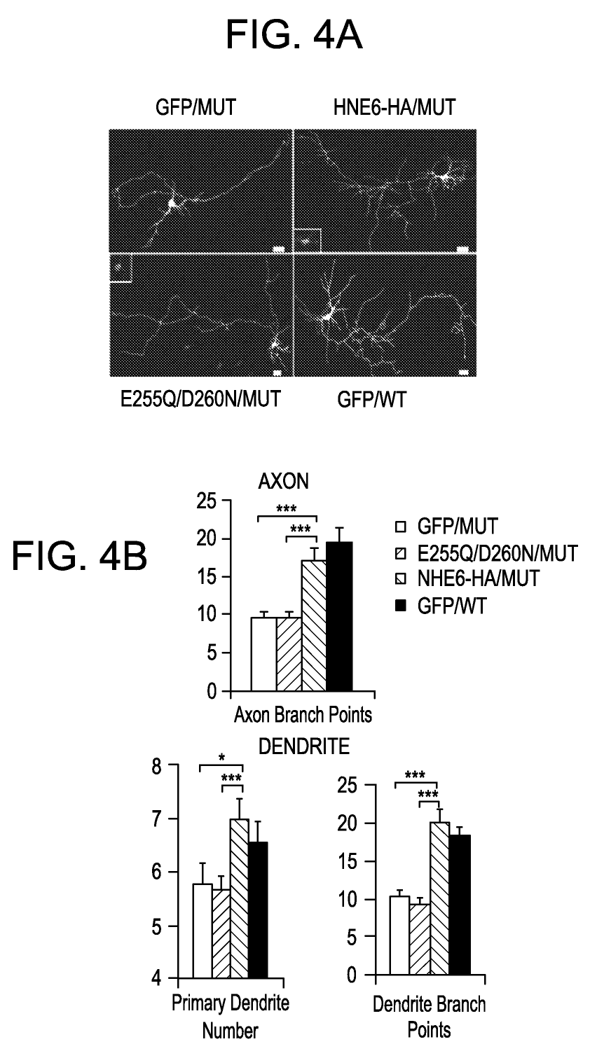

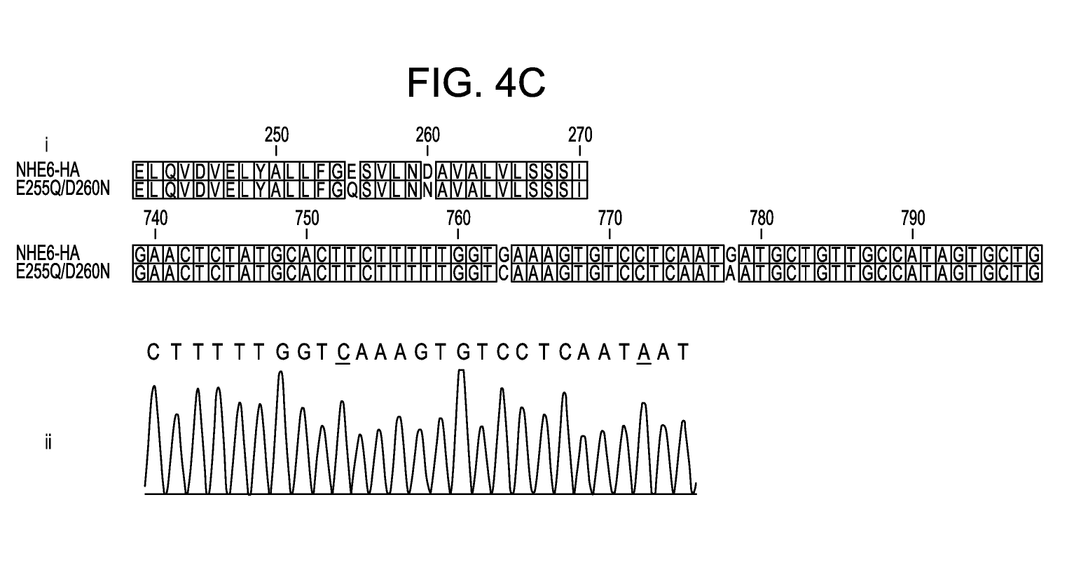

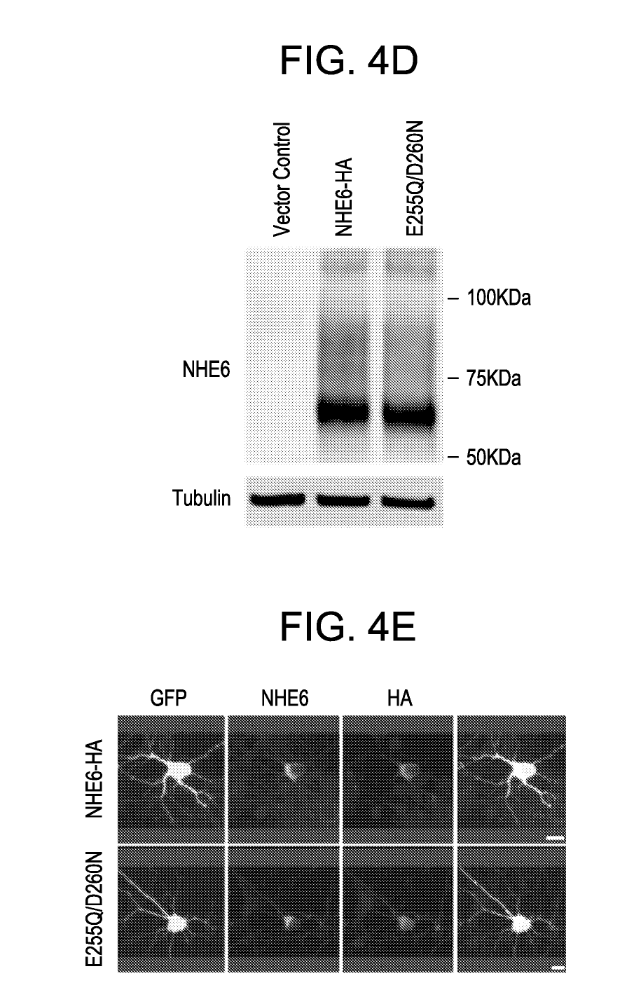

We next established a rescue assay to dissect the function of NHE6 in greater molecular detail. Defects in neuronal morphogenesis in NHE6-null neurons were rescued through transfection of expression constructs for the human full length NHE6 protein in these cultures (FIG. 4A). Quantification of this effect is shown in FIG. 4B, wherein the average level of axonal branching is reduced by 50% in the mutant (9.8+/−0.7 branch points in mutant vs 19.5+/−2 in wild-type, p<0.001) and dendritic branching is reduced by 43% in the mutant (10.6+/−0.7 branch points in mutant vs 18.5+/−1.2 in wild-type, p<0.001) in the GFP-alone transfected mutant neurons. The level of branching is normalized by transfection with the construct expressing the full-length human NHE6.0 transcript (17.3+/−1.5 in axon branch points and 20.4+/−1.5 in dendritic branch points in mutant cells). Overexpression of the full-length human NHE6.0 transcript also increases the primary dendrite number to the normal level.

To determine the functional domains of the NHE6 protein that are important for proper neuronal morphogenesis, we generated an NHE6 construct with mutations in the cation exchanger domain, an alteration known to impede proton transport (Xinhan et al., 2011) (FIG. 4C). This transport-deficient NHE6 protein was stable and appeared to traffic normally (FIGS. 4D and 4E). However, the transport-deficient NHE6 completely failed to rescue neuronal morphogenesis in the NHE6-null cultures (FIGS. 4A and 4B). Neurons transfected with the mutant construct continued to develop in a manner similar to mutant tissue, and without showing increases in cell death by analysis of pyknotic nuclei (data not shown). Impairments in dendritic and axonal branching were maintained in these cells despite stable protein expression and trafficking of this exchanger-deficient NHE6. These data support an interpretation that the endosomal proton leak function of NHE6 is required for the role of the protein in neuronal arborization.

### Example 4

### Nhe6-Null Mice have Normal Paired-Pulse Ratios, but Reduced Functional Connectivity

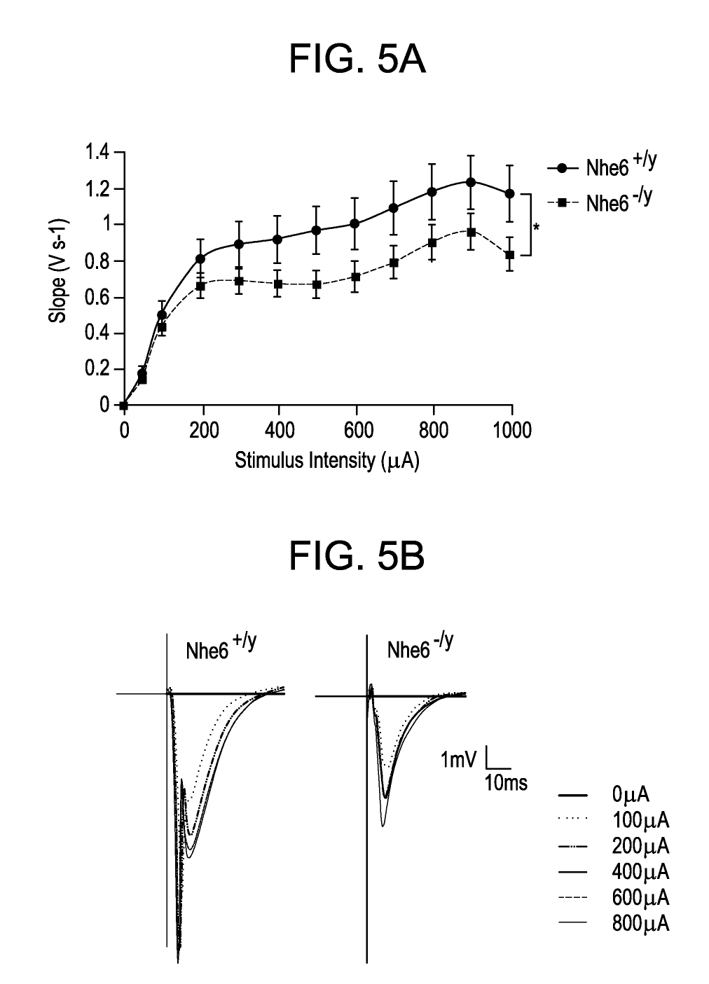

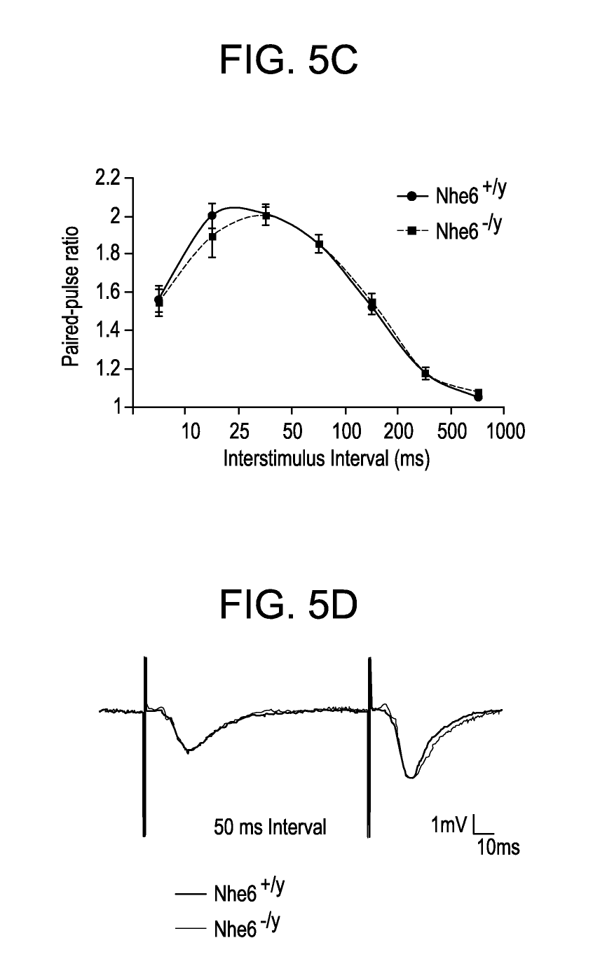

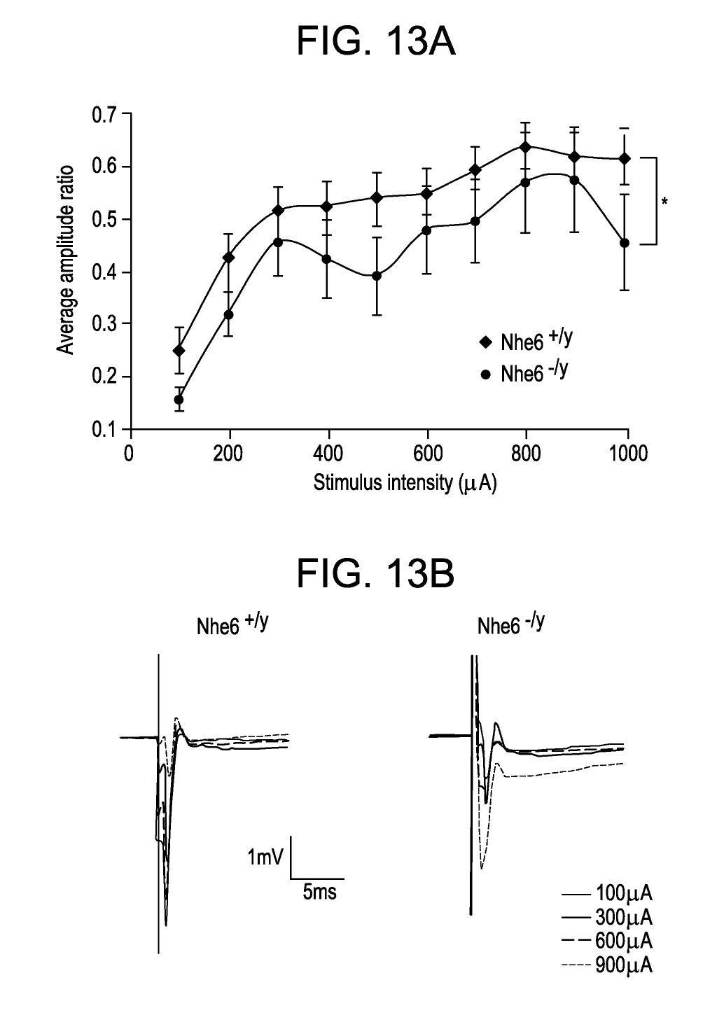

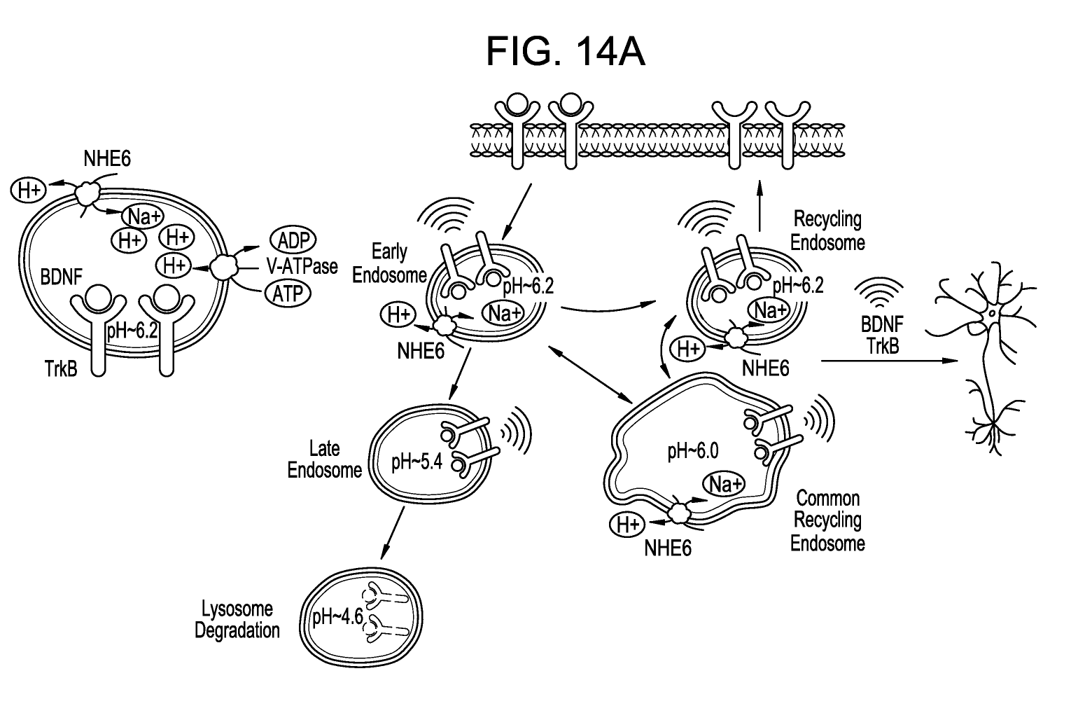

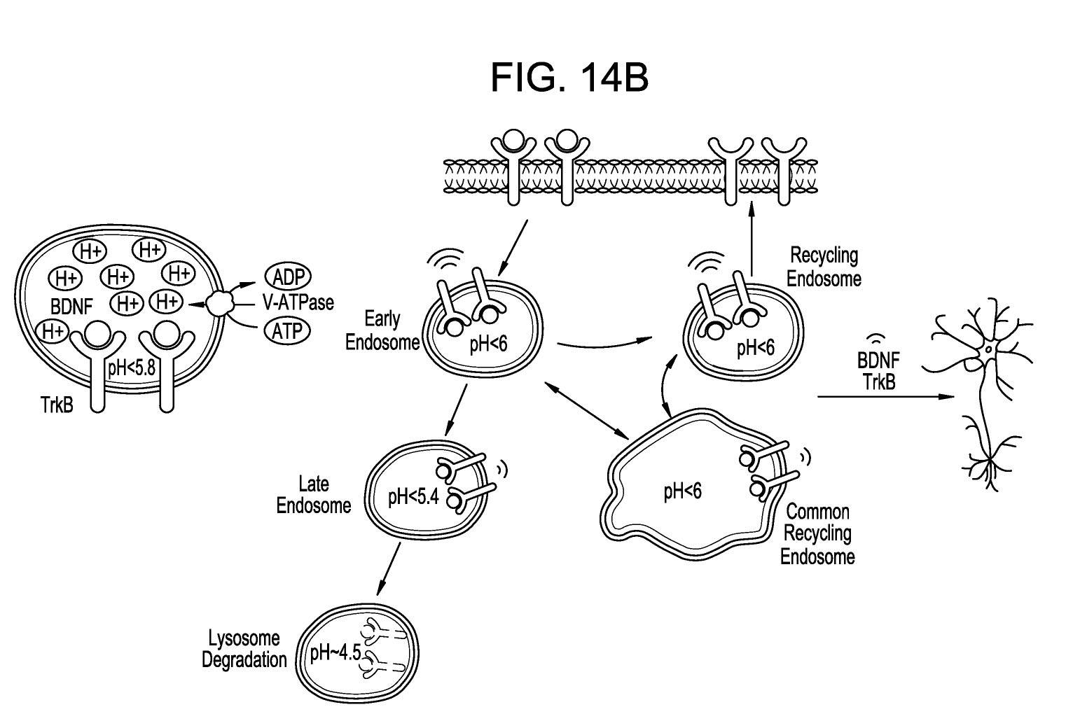

In order to understand further the functional consequences of NHE6 mutation on neuronal function, we compared extra-cellularly recorded synaptic field potentials in mutant versus wild-type animals in acute hippocampal slices. As demonstrated by the input/output curve, the extracellular synaptic potential was significantly reduced on average by 23.2% (p<0.0001) in the NHE6-mutants compared to control animals between postnatal days 14 to 21 (FIGS. 5A and 5B). This result is consistent with at least three models: first, that the synapses within the given circuit are individually weaker; second, there are fewer axons and/or branches activated by a given presynaptic stimulus, and third, that there are fewer functional synapses. Either or both of the last two interpretations are consistent with our prior observations of reduced axonal and dendritic branching. To test these possible models we further examined the properties of synapses in acute hippocampal slice preparations. As NHE6 protein staining appeared quantitatively more presynaptic than postsynaptic (FIGS. 2E and 2F), we first tested presynaptic function. We measured paired-pulse facilitation (PPF), a form of presynaptic short-term plasticity reflecting the synaptic vesicle probability of release over different time intervals (FIGS. 5C and 5D). The data show that mutant and wild-type synapses in hippocampal slices behaved identically, suggesting that the difference in the input/output curve is unlikely to be caused by difference in presynaptic release parameters. To examine if the reduced input/output curves in the NHE6 deficient mice was caused by a reduction in the number of axons, we analyzed the amplitude of the fiber volley in the presence of an AMPAR inhibitor. The fiber volley is directly related to the number of action potentials in stimulated presynaptic axons. In paired littermates we found a variable but significant decrease of 25% (p<0.001) in the fiber volley amplitude in the absence of NHE6 as compared to control (FIGS. 13A and 13B). The electrophysiological data support a model involving circuit defects with a reduction in active synapses due to a reduction of afferent input without evidence thus far of perturbed individual synapse function. Overall, these circuit defects (reduced synaptic strength and pre-synaptic fiber volley with intact paired-pulse facilitation) are consistent with the abnormalities in axonal and dendritic branching presented previously.

### Example 5

### Nhe6 Mutant Mice Display Reduced Synapse Number and Decreased Mature Spines

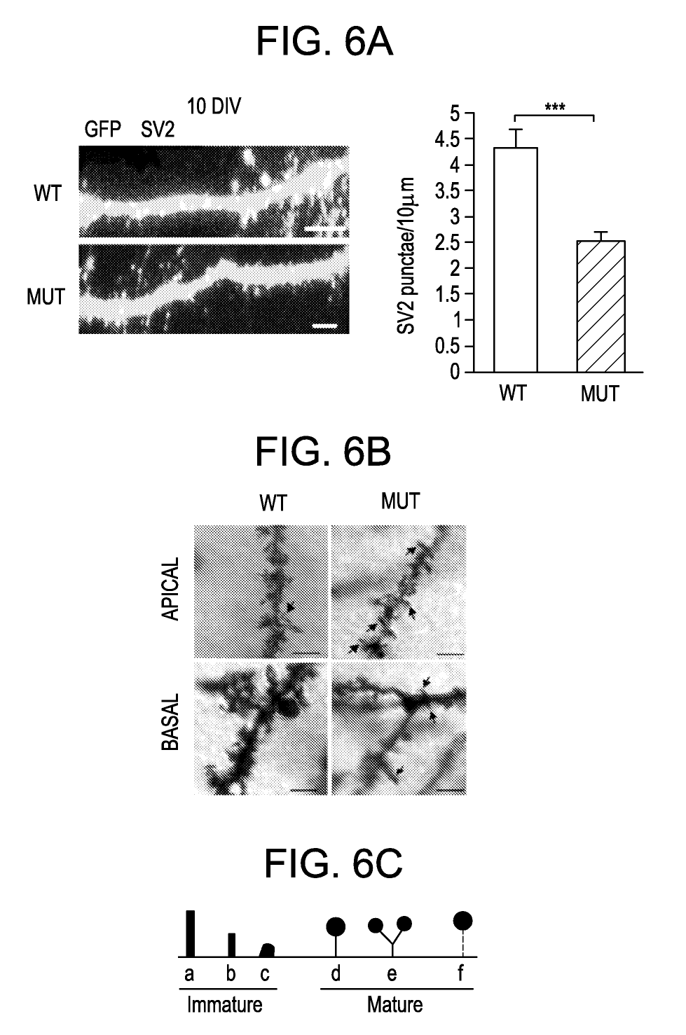

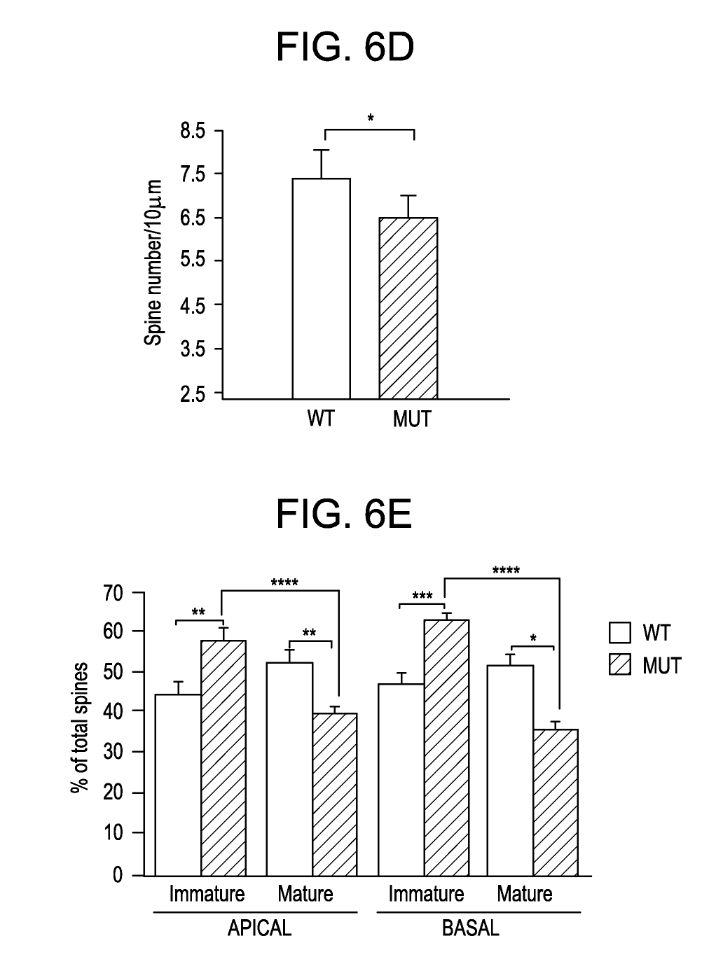

Reductions in axonal branches would predict corresponding reductions in synapses. To examine this hypothesis, we quantified the number of synapses and spines in NHE6 mutant mice. By quantifying SV2-positive punctae along the dendrites of GFP-transfected neurons in wild-type and mutant hippocampal cultures, we discovered that neurons in NHE6 mutant cultures displayed an approximately 42% decrease in SV2-positive presynapses per 10 μm of dendrite (4.3+/−0.4 SV2+ punctae in wild-type versus 2.5+/−0.2 in mutant, p<0.001) at 10 DIV (FIG. 6A). This observation was corroborated by the study of Golgi-Cox labeled spines in vivo at postnatal day 21. Using this method in CA1 hippocampal pyramidal neurons, we discovered a significant 12% decrease (p=0.03) in the total number of dendritic spines per 10 μm length (FIGS. 6B to 6E). We also found NHE6-null mice had a very significant decrease in the percentage of mature synapses both in basal dendrites (mutant=36.4+1.6% mature spines; control=52.3+2.5% mature spines, p=0.0009) and apical dendrites (mutant=40.2+1.8% mature spines; control=52.9+3.0 mature spines, p=0.0001) (FIG. 6F). In addition, we observed a highly significant increase in the percentage of immature synapses in the mutant animals compared to controls both in basal dendrites (mutant=63.6+1.6% immature spines; control=47.7+2.5% immature spines, p=0.000004) and apical dendrites (mutant=58.5+2.3% immature spines; control=45.1+2.8 immature spines, p=0.0005) dendrites. Therefore, the electrophysiology data suggesting reduced synaptic field strength due to decreases in synaptic number is consistent with our direct measures both in vitro and in vivo of reduced synapse number. Furthermore, the reduction in active synapses in NHE6-null mice correlates with an increase in the ratio of immature to mature synapses in the mutant animals compared to control animals. Altogether these data support the interpretation that NHE6 is required for proper circuit development in mouse hippocampus in vivo.

### Example 6

### Nhe6 Mutant Neurons Show Ectopic Low-pH Endosomes within Growing Axons and Dendrites

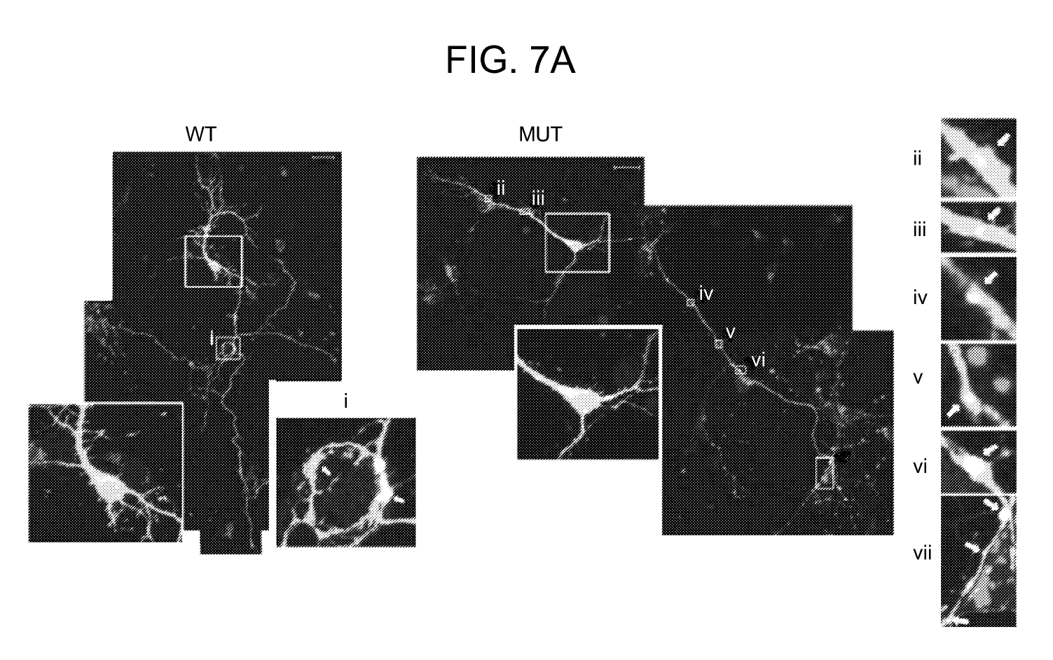

Our data are consistent with the hypothesis that NHE6 functions in the regulation of intra-endosomal pH within growing axons and dendrites, and in the absence of cation exchange, branching is impaired. As Na+/H+ exchangers are generally construed as passively transporting selective cations down their concentration gradients, we hypothesize NHE6 to be a “proton leak” channel for early endosomes. In the absence of NHE6, protons would be retained within these endosomes and an expanded distribution of acidic (i.e. low-pH endosomes) would be evident. We directly tested the above hypothesis by visualizing low-pH endomembranes within differentiating hippocampal neurons. Using the low-pH dye LysoTracker, we visualized low-pH endomembranes (pH<6.0) associated with cell soma that likely represent lysosomes. Intra-axonal LysoTracker-positive endomembranes were rarely identified in controls (FIG. 7Ai). Our results in controls are consistent with prior studies that have shown a proximal to distal gradient of low-pH endosomes. These studies have shown proximal intra-axonal endosomes to exhibit low luminal pH (pH<6.0) and distal intra-axonal endosomes representing higher luminal pH (pH>6.4) that would not be visualized by LysoTracker. In contrast to our control study, NHE6 mutant neurons showed an abundance of distally-placed LysoTracker-positive axonal and dendritic endosomes (FIG. 7A). Ectopic low-pH endosomes were observed along the axon, at branches of developing axons and dendrites and at distances of up to 150 μm and further. On average, low-pH endosomes were 74 μm from the soma in NHE6 mutant neurons as compared to 41 μm for control neurons (p<0.001). These data are consistent with abnormal acidification of endosomes in the absence of NHE6.

### Example 7

### TrkB and Phospho-Trk Levels are Reduced in Response to WAIF Signaling in the Absence of Nhe6

Our data taken together support a model wherein NHE6 is required for regulation of intra-endosomal proton concentration which is required for axon and dendritic branching. We next hypothesized that abnormally endosomal acidification may perturb endosomal signaling mechanisms relevant to neuronal arborization. Given the well-known role for BDNF/TrkB endosomal signaling in neuronal arborization, we set out to test the role of this signaling pathway in the pathophysiology and/or treatment for NHE6 mutations.

We initially investigated to see if NHE6 and TrkB protein, the BDNF receptor, co-localized to the same endosomes (FIG. 8A). We co-transfected GFP-TrkB expressing constructs with NHE6-HA expressing constructs. We discovered a very high degree of overlap of these two proteins in a distribution of punctate staining in the perinuclear area but also along growing axons and dendrites (FIG. 8A).

Next, we examined directly the levels of TrkB receptor in response to signaling. TrkB is well-known to be endocytosed upon binding BDNF and a significant degree of signaling has been proposed to occur within signaling endosomes upon endocytosis. Given the possibility of abnormal acidification of early endosomes in the absence of NHE6, we hypothesized that TrkB protein may show increased rates of degradation and consequently reduced signaling. To test this hypothesis, we examined TrkB receptor levels and phospho-Trk levels in response to BDNF signaling in NHE6 mutant and wild type hippocampal cultures at 4 DIV. Western blot analysis indicated that after adding 50 ng/ml of BDNF for 30 min, TrkB receptor level in mutant neurons was significantly reduced to 76.5% of the control (0.79+/−0.04 in mutant versus 1.03+/−0.06 in wild type where receptor levels are normalized to time of BDNF administration). The reduction effect in mutant neurons was maintained throughout 2 h of BDNF treatment (73.3% and 74.9% of wild type at 60 min and 2 h, respectively; p=8.35E-05, n=8 replicates). The induction of phospho-Trk was also significantly reduced in mutant after adding BDNF (induction was approximately 48% compared to control throughout the whole treatment duration; p=2.44E-08; n=4 replicates) (FIG. 8B). Collectively, these findings indicate that depletion of NHE6 has an effect on TrkB protein turnover and endosomal signaling, and these data suggest that the failures in dendrite and axon growth and branching in NHE6 mutants may be in part due to deficiencies in BDNF/TrkB signaling.

### Example 8

### Rescue of Branching Defects by Exogenous Administration of BDNF

Taken together our data support a model wherein due to loss of NHE6 there is an abnormal acidification of early endosomes resulting in impoverished branching as a result of decreases in BDNF/TrkB signaling. Given this model, we predicted that exogenous and high levels of BDNF may rescue the branching defects through enhancing TrkB signaling. To investigate the ability of exogenous BDNF to interact and improve the cellular phenotype seen, we added BDNF (50 ng/ml) to wild-type and mutant cultures at 2 DIV and examined cultures on day 5 for effects on axonal and dendritic branching associated with NHE6 mutation. We discovered that exogenous BDNF increased both axonal and dendritic branching in mutant cells to a level approaching wild-type levels (FIGS. 8C and 8D). We saw effects on both branching as well as an increase in the number of primary dendrites. In NHE6 mutant neurons exposed to exogenous BDNF, axonal branching increased from 16.7+/−1.3 branch points per cell to 27.7+/−2.0 branch points per cell (p<0.000013). Dendritic branching increased from 20.3+/−1.6 branches per cell to 34.4+/−2.4 branches per cell. Primary dendrites increased from an average of 6.5+/−0.4 dendrites to 8.7+/−0.4. Our data are consistent with the notion that BDNF/TrkB endosomal signaling is attenuated with loss of NHE6 yet this loss may be overcome through addition of exogenous levels of BDNF.

### OTHER EMBODIMENTS

Unless otherwise explained, all technical and scientific terms used herein have the same meaning as commonly understood by one of ordinary skill in the art to which this disclosure belongs. While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of treating, ameliorating, reversing, or slowing the progression of at least one symptom of a microcephaly associated autism disorder in a subject, said method comprising administering an agent that increases the level or activity of brain derived neurotrophic factor (BDNF) in the brain of said subject, thereby treating, ameliorating, or slowing the progression of at least one symptom of said microcephaly associated autism disorder in said subject.

2. A method of treating ameliorating, reversing, or slowing the progression at least one symptom of microcephaly associated autism disorder in a subject, said method comprising administering an agent that increases the level or activity of tyrosine related kinase B (TrkB) receptor in the brain of said subject, thereby treating, ameliorating, or slowing the progression of at least one symptom of said microcephaly associated autism disorder in said subject.

3. The method of any of claim 1 or 2, wherein microcephaly associated autism disorder is selected from the group consisting of Christianson syndrome, Angelman-like syndrome, and autism disorders with microcephaly.

4. The method of claim 3, wherein said microcephaly associated autism disorder is Christianson syndrome.

5. The method of any one of claims 1 to 4, wherein said subject has at least one mutation in the Nhe6 gene such that the gene encodes a mutant NHE6 protein.

6. The method of claim 5, wherein said mutant NHE6 protein has an E255Q mutation.

7. The method of claim 5, wherein said mutant NHE6 protein has a D260N mutation.

8. The method of any one of claims 1 to 7, wherein said agent is selected from the group consisting of BDNF, a BDNF agonist, a BDNF mimetic, a TrkB agonist, a cell expressing recombinant BDNF, a BDNF encoding recombinant nucleic acid molecule encapsidated within a recombinant virus, and an agent that decreases the acidity of endosomes.

9. The method of claim 8, wherein said agent is BDNF.

10. The method of claim 9, wherein said agent is recombinant BDNF.

11. The method of claim 8, wherein said agent is a BDNF agonist.

12. The method of claim 8, wherein said agent is a BDNF mimetic.

13. The method of claim 12, wherein said BDNF mimetic is a peptide mimetic comprising one or more mimetics selected from the group consisting of a tricyclic dimeric peptide, a dipeptide, a tripeptide, and a cyclic pentapeptide.

14. The method of claim 13, wherein said tripeptide is LM22A-1.

15. The method of claim 13, wherein said cyclic pentapeptide is cyclic pentapeptide 2.

16. The method of claim 12, wherein said BDNF mimetic is a chimeric neurotrophic factor.

17. The method of claim 12, wherein said BDNF mimetic is a non-peptide mimetic.

18. The method of claim 12, wherein said BDNF mimetic is a small molecule mimetic.

19. The method of claim 18, wherein said small molecule mimetic is selected from the group consisting of LM22A-2, LM22A-3, and LM22A-4.

20. The method of claim 12, wherein said BDNF mimetic is deoxygedunin.

21. The method of claim 12, wherein said BDNF mimetic is 7, 8-dihydroxyflavone.

22. The method of claim 11, wherein said BDNF agonist is a TrkB activating antibody.

23. The method of claim 11, wherein said agent is a BDNF agonist selected from the group consisting of neurotrophin-4, neurotrophin-5, N-acetylserotonin, and 4′-dimethylamino-7, 8-dihydroxyflavone.

24. The method of claim 8, wherein said agent is a cell expressing BDNF.

25. The method of claim 24, wherein said cell is an induced pluripotent stem cell (iPSC).

26. The method of claim 8, wherein said recombinant virus is selected from the group consisting of recombinant adeno-associated virus (AAV), recombinant retrovirus, recombinant lentivirus, recombinant poxvirus, recombinant rabies virus, recombinant pseudo-rabies virus, and recombinant herpes simplex virus, and human immunodeficiency virus (HIV).

27. The method of claim 8, wherein said agent is an agent that decreases the acidity of endosomes.

28. The method of claim 27, wherein said agent that decreases the acidity of endosomes is selected from the group consisting of amantadine, amiodarone, ammonium chloride, azithromycin, bafilomycin A1, benzolactone enamides, bepridil, diphyllin, indolyls, macrolactones, monensin, nigericin, plecomacrolides, quinolines, or sulfonamides.

29. The method of claim 28, wherein said agent is a benzolactone enamide selected from the group consisting of salicylihalamide, lobatamide, apicularen, oximidine, and cruentaren.

30. The method of claim 28, wherein said agent is an indole derivative of bafilomycin.

31. The method of claim 30, wherein said indole derivative of bafilomycin is INDOL0.

32. The method of claim 28, wherein said agent is a macrolactone selected from the group consisting of archazolid and azithromycin.

33. The method of claim 28, wherein said agent is a plecomacrolide selected from the group consisting of bafilomycin A1 and concanamycin.

34. The method of claim 28, wherein said agent is a quinoline selected from the group consisting of amodiaquine, chloroquine, and hydroxychloroquine.

35. The method of claim 34, wherein said quinoline is chloroquine.

36. The method of claim 28, wherein said agent is a sulfonamide selected from the group consisting of 16D2 and 16D10.

37. The method of any one of claims 1 to 36, wherein said symptom is selected from the group consisting of intellectual disability, an intelligence quotient (IQ) of 70 or less, epilepsy, inability to speak, craniofacial dysmorphology, ataxia, difficulty with adaptive skills, difficulty walking, inability to walk, ophthalmoplegia, brain atrophy, autistic symptoms, and retinitis pigmentosa.

38. The method of claim 37, wherein said agent treats, ameliorates, reverses, or slows progression toward said inability to speak.

39. The method of claim 37, wherein said agent treats, ameliorates, reverses, or slows progression of said intellectual disability and increases said IQ.

40. The method of claim 37, wherein said agent decreases said epilepsy.

41. The method of claim 37, wherein said agent decreases said ataxia.

42. The method of claim 37, wherein said agent treats, ameliorates, reverses, or slows progression of said brain atrophy.

43. The method of claim 37, wherein said agent increases said adaptive skills.

44. The method of claim 37, wherein said agent treats, ameliorates, reverses, or slows progression of said difficulty walking.

45. The method of claim 37, wherein said agent treats, ameliorates, reverses, or slows progression toward said inability to walk.

46. The method of any one of claims 1 to 45, further comprising, prior to administering said agent, testing said subject for microcephaly.

47. The method of claim 46, wherein said testing comprises comparing the head circumference of said subject to that of a control subject or reference value.

48. The method of any one of claims 1 to 47, further comprising, prior to administering said agent, testing said subject for the presence of a microcephaly associated autism disorder selected from the group consisting of Christianson syndrome, Angelman-like syndrome, and autism with microcephaly.

49. The method of claim 48, wherein said testing comprises testing the presence of one or more mutations in the Nhe6 gene in said subject relative to a control subject or reference sequence.

50. The method of claim 49, wherein said control subject is a subject without a microcephaly associated autism disorder.

51. The method of any one of claims 48 to 50, wherein said testing comprises a genomic sequencing assay, polymerase chain reaction assay, fluorescence in situ hybridization assay, or an immunoassay.

52. The method of any one of claims 1 to 51, further comprising, prior to administering said agent, assaying in said subject one or more symptoms selected from the group consisting of intellectual disability, delayed development, sleep disturbance, epilepsy, jerky movements (especially hand-flapping), social-interaction difficulties, communication challenges, repetitive behaviors, ataxia, craniofacial dysmorphology, difficulty with adaptive skills, difficulty standing or walking, inability to walk, ophthalmoplegia, brain atrophy, retinitis pigmentosa, severe limitation of speech and language, easily provoked laughter, a happy demeanor with frequent smiling or spontaneous laughter, impaired ocular movement, and autistic behaviors as measured by the autism diagnostic observation schedule (ADOS) and/or the autism diagnostic interview-revised (ADI-R).

53. The method of any one of claims 1 to 52, further comprising, prior to administering said agent, assaying said subject one or more symptoms selected from a low level or activity of BDNF and a low level or activity of TrkB.

54. The method of any one of claims 1 to 53, further comprising, subsequent to administering said agent, assaying in said subject one or more symptoms selected from the group consisting of intellectual disability, delayed development, sleep disturbance, epilepsy, jerky movements (especially hand-flapping), social-interaction difficulties, communication challenges, repetitive behaviors, ataxia, craniofacial dysmorphology, difficulty with adaptive skills, difficulty standing or walking, inability to walk, ophthalmoplegia, brain atrophy, retinitis pigmentosa, severe limitation of speech and language, easily provoked laughter, a happy demeanor with frequent smiling or spontaneous laughter, impaired ocular movement, and autistic behaviors as measured by the autism diagnostic observation schedule (ADOS) and/or the autism diagnostic interview-revised (ADI-R).

55. The method of any one of claims 1 to 54, further comprising, subsequent to administering said agent, assaying in said subject one or more symptoms selected from a low level or activity of BDNF and a low level or activity of TrkB.

56. The method of claim 54 or 55, wherein said subject demonstrates improvement in one or more of said symptoms relative to a pre-treatment value.

57. The method of any one of claims 1 to 56, wherein said subject is a newborn.

58. The method of any one of claims 1 to 56, wherein said subject is 1 day to 30 years of age.

59. The method of claim 58, wherein said subject is 1 day to 15 years of age.

60. The method of claim 59, wherein said subject is 1 day to 10 years of age.

61. The method of any one of claims 1 to 60, wherein said subject does not have one or more conditions selected from the group consisting of Alzheimer's disease, Huntington's disease, Parkinson's disease, Rett syndrome, traumatic brain injury, spinal cord injury, age-associated neuronal degeneration, excitotoxicity, stroke, neuropathic pain, depression, obesity, bipolar disorder, aggression, or substance abuse.

62. The method of any one of claims 1 to 61, wherein said subject does not have one or more conditions selected from the group consisting of hepatitis C, chronic fatigue syndrome, viral infection, influenza, bacterial infection, middle ear infection, strep throat, pneumonia, typhoid, bronchitis, urinary tract infection, malaria, fungal infection, sinusitis, multiple sclerosis, arrhythmia, ventricular arrhythmia, ventricular tachycardia, ventricular fibrillation, angina, atrial fibrillation, hypertension, metabolic alkalosis, hypochloremia, cancer, osteoporosis, bone lytic diseases, ischemia, silent ischemia, gastric disorders, and osteoclast hyperactivity.


SEQUENCE LISTING (10 sequences)

SEQ ID NO: 1 - DNA, 27 bp, Mus musculus
gggtgggattagataaatgcctgctct

SEQ ID NO: 2 - DNA, 24 bp, Mus musculus
aacagctgtggagggatatgtgct

SEQ ID NO: 3 - DNA, 24 bp, Mus musculus
agctggctttgcgcatggagcatt

SEQ ID NO: 4 - DNA, 58 bp, Homo sapiens
tctatgcacttctttttggtcaaagtgtcctcaataatgctgttgccatagtgctgtc

SEQ ID NO: 5 - DNA, 58 bp, Homo sapiens
gacagcactatggcaacagcattattgaggacactttgaccaaaaagaagtgcataga

SEQ ID NO: 6 - protein, 30 aa, Homo sapiens
ELQVDVELYALLFGESVLNDAVAIVLSSSI

SEQ ID NO: 7 - protein, 30 aa, Homo sapiens
ELQVDVELYALLFGQSVLNNAVAIVLSSSI

SEQ ID NO: 8 - DNA, 60 bp, Homo sapiens
gaactctatgcacttctttttggtgaaagtgtcctcaatgatgctgttgccatagtgctg

SEQ ID NO: 9 - DNA, 60 bp, Homo sapiens
gaactctatgcacttctttttggtcaaagtgtcctcaataatgctgttgccatagtgctg

SEQ ID NO: 10 - DNA, 27 bp, Homo sapiens
ctttttggtcaaagtgtcctcaataat